(19) United States
(12) Patent Application Publication
(10) Pub. No.: US 2020/0222354 A1
(43) Pub. Date: Jul. 16, 2020

(54) PHARMACEUTICAL COMPOSITIONS CONTAINING DIMETHYL FUMARATE

(71) Applicant: Biogen MA Inc., Cambridge, MA (US)

(72) Inventors: David Goldman, Medfield, MA (US); Katherine Dawson, Westwood, MA (US); Ajay Nirula, Westlake Village, CA (US)

(73) Assignee: Biogen MA Inc., Cambridge, MA (US)

(21) Appl. No.: 16/826,938

(22) Filed: Mar. 23, 2020

Related U.S. Application Data
(63) Continuation of application No. 14/679,716, filed on Apr. 6, 2015.
Application No. 14/679,716 is a continuation of application No. 13/760,916, filed on Feb. 6, 2013, now abandoned.
(60) Provisional application No. 61/723,048, filed on Nov. 6, 2012.
(60) Provisional application No. 61/625,621, filed on Apr. 17, 2012.
(60) Provisional application No. 61/596,202, filed on Feb. 7, 2012.

(51) Int. Cl. A61K 31/225; A61K 31/60; A61K 45/06; A61K 9/20; A61K 9/28; A61K 31/616; A61K 9/48; A61K 9/16

(52) U.S. Cl. CPC A61K 31/225; A61K 31/60; A61K 45/06; A61K 9/2054; A61K 9/2833; A61K 31/616; A61K 9/2072; A61K 9/4808; A61K 9/16; A61K 9/2846

(57) ABSTRACT

Provided herein are compositions containing compounds, or pharmaceutically acceptable salts, that metabolize to monomethyl fumarate with certain pharmacokinetic parameters and methods for treating, prophylaxis, or amelioration of neurodegenerative diseases including multiple sclerosis using such compositions in a subject, wherein if the compositions contain dimethyl fumarate, the total amount of dimethyl fumarate in the compositions ranges from about 43% w/w to about 95% w/w.

### BRIEF SUMMARY OF THE INVENTION

Provided herein are compositions containing compounds, or pharmaceutically acceptable salts, that metabolize to monomethyl fumarate (MMF) and methods for treating, prophylaxis, or amelioration of neurodegenerative diseases including multiple sclerosis using such compositions in a subject. In one embodiment, the compound that metabolizes to MMF is dimethyl fumarate (DMF).

Another embodiment is a method of treating, prophylaxis, or amelioration of neurogenerative diseases including multiple sclerosis, comprising administering to a subject in need thereof a composition containing a compound, or a pharmaceutically acceptable salt thereof, that metabolizes to MMF wherein said administering the composition provides one or more of the following pharmacokinetic parameters: (a) a mean plasma MMF Tₘₐₓ of from about 1.5 hours to about 3.5 hours; (b) a mean plasma MMF Cₘₐₓ ranging from about 1.03 mg/L to about 3.4 mg/L; (c) a mean plasma MMF AUCₒᵥₑᵣₐₗₗ ranging from about 4.81 h·mg/L to about 11.2 h·mg/L; (d) a mean plasma MMF AUC₀₋₁₂ ranging from about 2.4 h·mg/L to about 5.5 h·mg/L; and (e) a mean AUC_{0-infinity} ranging from about 2.4 h·mg/L to about 5.6 h·mg/L.

One embodiment is a composition comprising DMF and an excipient, wherein a total amount of DMF in the composition ranges from about 43% w/w to about 95% w/w.

Another embodiment is a method of making a composition comprising combining about 43% w/w to about 95% w/w DMF, about 3.5% w/w to about 55% w/w of one or more fillers, about 0.2% w/w to about 20% w/w of one or more disintegrants, about 0.1% w/w to about 90.0/% w/w of one or more glidants, and about 0.1% w/w to about 3.0% w/w of one or more lubricants to form the composition.

A further embodiment is a composition comprising DMF and one or more excipients, wherein about 80 (e.g., 97%) or higher of the DMF has a particle size of 250 microns or less.

An additional embodiment is a composition comprising DMF, wherein the composition is in the form of coated microtablets. Each uncoated microtablet contains a total amount of DMF of about 43% w/w to about 95% w/w (e.g., about 50% w/w to about 80% w/w). Patients administered the composition exhibit a mean plasma MMF Tₘₐₓ of from about 1.5 hours to about 3.5 hours.

One embodiment is a capsule comprising a composition in the form of microtablets comprising DMF, wherein the total amount of DMF in each uncoated microtablet ranges from about 43% w/w to about 95% w/w and the microtablet has a tensile strength ranging from about 0.5 MPa to about 5 MPa at an applied pressure ranging from about 25 MPa to about 200 MPa. Compacts (e.g., 10 mm cylindrical compacts) made with identical ingredients as the microtablets (i.e., the only difference between a microtablet and a compact is the shape) display a tensile strength of equal or greater than 1.5 MPa (e.g., 2.0-5.0 MPa) at an applied pressure of about 100 MPa. Such corresponding compacts have a tensile strength that is similar or higher than compacts that are made with 42% w/w or lower amount of DMF.

Another embodiment is microtablets comprising: DMF in the range of about 43% w/w to about 95% w/w, a total amount of filler in the range of about 3.5% w/w to about 55% w/w, a total amount of disintegrant in the range of about 0.2% w/w to about 20% w/w, a total amount of glidant in the range of about 0.1% w/w to about 9.0% w/w; and a total amount of lubricant in the range of about 0.1% w/w to about 3.0% w/w; wherein the microtablet has a tensile strength ranging from about 0.5 MPa to about 5 MPa at an applied pressure ranging from about 25 MPa to about 200 MPa and the corresponding compact has a tensile strength of equal or greater than 1.5 MPa (e.g., 2.0-5.0 MPa) at an applied pressure of about 100 MPa.

A further embodiment is a method of making a microtablet comprising DMF, wherein the amount of DMF in the uncoated microtablet is about 43% w/w to about 95% w/w and the corresponding compact has a tensile strength of equal or greater than 2.0 MPa (e.g., 2.0-5.0 MPa) at an applied pressure of about 100 MPa.

Other embodiments are methods for treating, prophylaxis, or amelioration of neurodegenerative diseases including multiple sclerosis using the compositions according to the present invention in a subject in combination with one or more non-steroidal anti-inflammatory drugs (e.g., aspirin).

### BRIEF DESCRIPTION OF THE DRAWINGS

FIG. 1 depicts a comparison of tensile strengths (MPa) of compacts containing 42% w/w and 65% w/w of DMF formed at different applied or compaction pressures (MPa).

FIG. 2 depicts a comparison of tensile strengths (MPa) of compacts containing 42% w/w, 60% w/w, 65% w/w, and 70% w/w of DMF formed at different applied or compaction pressures (MPa).

FIG. 3 depicts a comparison of tensile strengths (MPa) of compacts containing 65% w/w, 95% w/w, and 99.5% w/w of DMF formed at different applied or compaction pressures (MPa).

### DETAILED DESCRIPTION OF THE INVENTION

### Definitions

As used herein, “a” or “an” means one or more unless otherwise specified.

Open terms such as “include,” “including,” “contain,” “containing” and the like mean “comprising.”

The term “treating” refers to administering a therapy in an amount, manner, or mode effective to improve a condition, symptom, or parameter associated with a disorder.

The term “prophylaxis” or the term “ameliorating” refers to preventing a disorder or preventing progression of a disorder, to either a statistically significant degree or to a degree detectable to one skilled in the art.

The term “or” can be conjunctive or disjunctive.

The term “placebo” refers to a composition without active agent (e.g., DMF). Placebo compositions can be prepared by known methods, including those described herein.

The term “compact” means a compressed composition comprising DMF and one or more excipients. The DMF and excipients can be homogeneously or heterogeneously mixed in the compact.

The term “microtablet” means a compact in the form of a small (micro) tablet of about 1 mm to about 3 mm in diameter (excluding any coating) that comprises DMF and one or more excipients. The DMF and excipients can be homogeneously or heterogeneously mixed in the microtablet.

The term “coated microtablet” means a microtablet that is fully or partially coated by one or more coatings.

Unless otherwise specified (e.g., in Table 2 below), the term “% w/w” is the percent of an ingredient in a composition (e.g., a microtablet) excluding the weight of any coating component(s) (e.g., copolymer(s) forming an enteric coating) fully or partially coating the microtablet.

In some embodiments, the invention contemplates numerical ranges. Numerical ranges include the range endpoints. Additionally, when a range is provided, all subranges and individual values therein are present as if explicitly written out.

The term “alkyl” as employed herein by itself or as part of another group refers to both straight and branched chain radicals of up to 24 carbons. Alkyl groups include straight-chained and branched C₁-C₂₄ alkyl groups, e.g., C₁-C₁₀ alkyl groups. C₁-C₁₀ alkyl groups include methyl, ethyl, propyl, isopropyl, butyl, isobutyl, sec-butyl, tert-butyl, pentyl, isopentyl, neopentyl, hexyl, isohexyl, 3-methylpentyl, 2,2-dimethylbutyl, 2,3-dimethylbutyl, heptyl, 1-methylhexyl, 2-ethylhexyl, 1,4-dimethylpentyl, octyl, nonyl, and decyl. Unless otherwise indicated, all alkyl groups described herein include both unsubstituted and substituted alkyl groups. Further, each alkyl group can include its deuterated counterparts.

The term “aryl” as employed herein by itself or as part of another group refers to monocyclic, bicyclic, or tricyclic aromatic groups containing from 5 to 50 carbons in the ring portion. Aryl groups include C₁₋₂₄ aryl, e.g., phenyl, p-tolyl, 4-methoxyphenyl, 4-(tert-butoxy)phenyl, 3-methyl-4-methoxyphenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 3-amino-naphthyl, 2-methyl-3-amino-naphthyl, 6-amino-2-naphthyl, 4,6-dimethoxy-2-naphthyl, indanyl, biphenyl, phenanthryl, anthryl, and acenaphthyl. Unless otherwise indicated, all aryl groups described herein include both unsubstituted and substituted aryl groups.

Optional substituents on the alkyl group include one or more substituents independently selected from halogen, hydroxyl, carboxyl, amino, nitro, or cyano.

Optional substituents on the aryl group include one or more substituents independently selected from alkyl, alkoxy, halogen, hydroxyl, or amino.

Halogen groups include fluorine, chlorine, bromine, and iodine.

Some of the compounds of the present invention may exist as stereoisomers including optical isomers. The invention includes all stereoisomers and both the racemic mixtures of such stereoisomers as well as the individual enantiomers that may be separated according to methods that are well-known to those of ordinary skill in the art.

### Introduction

Multiple sclerosis (MS) is an autoimmune disease with the autoimmune activity directed against central nervous system (CNS) antigens. The disease is characterized by inflammation in parts of the CNS, leading to the loss of the myelin sheathing around neuronal axons (demyelination), axonal loss, and the eventual death of neurons, oligodenrocytes and glial cells. For a comprehensive review of MS and current therapies, see, e.g., McAlpine's Multiple Sclerosis, by Alastair Compston et al., 4th edition, Churchill Livingstone Elsevier, 2006.

DMF has been studied for an oral treatment of MS. In two recently completed Phase III studies, BG-12, which contains DMF as the only active ingredient, significantly improved clinical and neuroradiologic endpoints versus placebo when dosed at 240 mg of DMF twice a day (BID) or 240 mg of DMF thrice a day (TID). Patients in both Phase III studies were administered capsules containing 120 mg of DMF. That means patients had to take 4 or 6 capsules a day, which presents a burden to the patients and a challenge for patient compliance. To promote treatment adherence, it is desirable to decrease the number of capsules a patient has to take per day by increasing the drug load of the dosage form (e.g., a capsule).

It has been found that a composition comprising a total amount of DMF ranging from about 43% w/w to about 95% w/w (e.g., from about 50% w/w to about 80% w/w or from about 60% w/w to about 70% w/w) and one or more excipients formulated in such a manner that about 160 mg of DMF to about 500 mg of DMF (e.g., about 240 mg to about 480 mg DMF) can be included in a single dosage form that can be administered, for example, once per day (QD), BID, or TID. For example, a capsule (e.g., size 0) can contain about 240 mg of DMF. As another example, a capsule can contain about 480 mg of DMF.

In general, when the drug load (or weight percent of an active ingredient) of a solid oral dosage form (e.g., a tablet or a microtablet) is significantly increased, the weight percent of the excipient(s) must decrease (especially if the size of the solid oral dosage form remains the same). The solid oral dosage form often becomes unstable due to the decrease in the amount of excipient(s), e.g., binders, that function to hold all the components together in a cohesive mix. It is unexpected that increasing the amount of DMF (e.g., from 120 mg to 240 mg) and decreasing the amount of binder, while keeping the size of the solid oral dosage form (e.g., capsule size) to be the same, the strength or integrity of solid dosage form does not suffer.

Additionally, it has been found that a composition containing a compound, or a pharmaceutically acceptable salt thereof, that metabolizes to MMF wherein said administering the composition provides one or more of the following pharmacokinetic parameters: (a) a mean plasma MMF Tₘₐₓ of from about 1.5 hours to about 3.5 hours; (b) a mean plasma MMF Cₘₐₓ ranging from about 1.03 mg/L to about 3.4 mg/L; (c) a mean plasma MMF AUCₒᵥₑᵣₐₗₗ ranging from about 4.81 h·mg/L to about 11.2 h·mg/L; (d) a mean plasma MMF AUC₀₋₁₂ ranging from about 2.4 h·mg/L to about 5.5 h·mg/L; and (e) a mean AUC_{0-infinity} ranging from about 2.4 h·mg/L to about 5.6 h·mg/L can be administered to a subject in need thereof to treat, prophylaxis, or amelioration of multiple sclerosis.

All of the various aspects, embodiments, and options disclosed herein can be combined in any and all variations. The compositions and methods provided are exemplary and are not intended to limit the scope of the claimed embodiments.

### DISCUSSION

In one embodiment, a method of treating, prophylaxis, or amelioration of multiple sclerosis, comprising administering to a subject in need thereof a composition containing a compound, or a pharmaceutically acceptable salt thereof, that metabolizes to MMF wherein said administering the composition provides one or more of the following pharmacokinetic parameters: (a) a mean plasma MMF Tₘₐₓ of from about 1.5 hours to about 3.5 hours; (b) a mean plasma MMF Cₘₐₓ ranging from about 1.03 mg/L to about 3.4 mg/L; (c) a mean plasma MMF AUCₒᵥₑᵣₐₗₗ ranging from about 4.81 h·mg/L to about 11.2 h·mg/L; (d) a mean plasma MMF AUC₀₋₁₂ ranging from about 2.4 h·mg/L to about 5.5 h·mg/L; and (e) a mean AUC_{0-infinity} ranging from about 2.4 h·mg/L to about 5.6 h·mg/L.

In a further embodiment, the composition is orally administered to the subject in need thereof.

In some embodiments, the compound that metabolizes to MMF is DMF.

In some embodiments, the compound that metabolizes to MMF is a compound of Formula I:

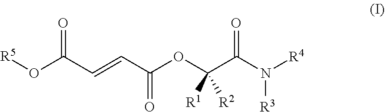

or a pharmaceutically acceptable salt thereof, wherein

R¹ and R² are independently chosen from hydrogen, C₁₋₆ alkyl, and substituted C₁₋₆ alkyl;

R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl; and

R⁵ is chosen from methyl, ethyl, and C₃₋₆ alkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl;

with the proviso that when R⁵ is ethyl; then R³ and R⁴ are independently chosen from hydrogen, C₁₋₆ alkyl, and substituted C₁₋₆ alkyl.

In certain embodiments of a compound of Formula (I), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl. In certain embodiments, each substituent group is independently chosen from —OH, and —COOH.

In certain embodiments of a compound of Formula (I), each substituent group is independently chosen from —O, C₁₋₄ alkyl, and —COOR¹¹ wherein R¹¹ is chosen from hydrogen and C₁₋₄ alkyl.

In certain embodiments of a compound of Formula (I), each of R¹ and R² is hydrogen.

In certain embodiments of a compound of Formula (I), one of R¹ and R² is hydrogen and the other of R¹ and R² is C₁₋₄ alkyl.

In certain embodiments of a compound of Formula (I), one of R¹ and R² is hydrogen and the other of R¹ and R² is chosen from methyl, ethyl, n-propyl, isopropyl, n-butyl, isobutyl, sec-butyl, and tert-butyl.

In certain embodiments of a compound of Formula (I), one of R¹ and R² is hydrogen and the other of R¹ and R² is methyl.

In certain embodiments of a compound of Formula (I), R³ and R⁴ are independently chosen from hydrogen and C₁₋₄ alkyl.

In certain embodiments of a compound of Formula (I), R³ and R⁴ are independently chosen from hydrogen and C₁₋₄ alkyl.

In certain embodiments of a compound of Formula (I), R³ and R⁴ are independently chosen from hydrogen, methyl, and ethyl.

In certain embodiments of a compound of Formula (I), each of R³ and R⁴ is hydrogen; in certain embodiments, each of R³ and R⁴ is methyl; and in certain embodiments, each of R³ and R⁴ is ethyl.

In certain embodiments of a compound of Formula (I), R³ is hydrogen; and R⁴ is chosen from C₁₋₄ alkyl, substituted C₁₋₄ alkyl wherein the substituent group is chosen from ═O, —OR¹¹, —COOR¹¹, and —NR¹¹₂, wherein each R¹¹ is independently chosen form hydrogen and C₁₋₄ alkyl.

In certain embodiments of a compound of Formula (I), R³ is hydrogen; and R⁴ is chosen from C₁₋₄ alkyl, benzyl, 2-methoxyethyl, carboxymethyl, carboxypropyl, 1,2,4-thiadoxolyl, methoxy, 2-methoxycarbonyl, 2-oxo(1,3-oxazolidinyl), 2-(methylethoxy)ethyl, 2-ethoxyethyl, (tert-butyloxycarbonyl)methyl, (ethoxycarbonyl)methyl, carboxymethyl, (methylethyl)oxycarbonylmethyl, and ethoxycarbonylmethyl.

In certain embodiments of a compound of Formula (I), R³ and R⁴ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (I), R³ and R⁴ together with the nitrogen to which they are bonded form a ring chosen from a C₅ heterocycloalkyl, substituted C₅ heterocycloalkyl, C₅ heteroaryl, and substituted C₅ heteroaryl ring. In certain embodiments of a compound of Formula (I), R³ and R⁴ together with the nitrogen to which they are bonded form a ring chosen from a C₆ heterocycloalkyl, substituted C₆ heterocycloalkyl, C₆ heteroaryl, and substituted C₆ heteroaryl ring. In certain embodiments of a compound of Formula (I), R³ and R⁴ together with the nitrogen to which they are bonded form a ring chosen from piperazine, 1,3-oxazolidinyl, pyrrolidine, and morpholine ring.

In certain embodiments of a compound of Formula (I), R³ and R⁴ together with the nitrogen to which they are bonded form a C₅₋₁₀ heterocycloalkyl ring.

In certain embodiments of a compound of Formula (I), R⁵ is methyl.

In certain embodiments of a compound of Formula (I), R⁵ is ethyl.

In certain embodiments of a compound of Formula (I), R⁵ is C₃₋₆ alkyl.

In certain embodiments of a compound of Formula (I), R⁵ is chosen from methyl, n-propyl, isopropyl, n-butyl, sec-butyl, isobutyl, and tert-butyl.

In certain embodiments of a compound of Formula (I), R⁵ is chosen from methyl, ethyl, n-propyl, isopropyl, n-butyl, sec-butyl, isobutyl, and tert-butyl.

In certain embodiments of a compound of Formula (I), one of R¹ and R² is hydrogen and the other of R¹ and R² is C₁₋₆ alkyl; R³ is hydrogen; R⁴ is chosen from hydrogen, C₁₋₆ alkyl, and benzyl.

In certain embodiments of a compound of Formula (I), one of R¹ and R² is hydrogen and the other of R¹ and R² is C₁₋₆ alkyl; R³ is hydrogen; R⁴ is chosen from hydrogen, C₁₋₆ alkyl, and benzyl; and R⁵ is methyl.

In certain embodiments of a compound of Formula (I), one of R¹ and R² is hydrogen and the other of R¹ and R² is chosen from hydrogen and C₁₋₆ alkyl; and each of R³ and R⁴ is C₁₋₆ alkyl.

In certain embodiments of a compound of Formula (I), one of R¹ and R² is hydrogen and the other of R¹ and R² is chosen from hydrogen and C₁₋₆ alkyl; each of R³ and R⁴ is C₁₋₆ alkyl; and R⁵ is methyl. In certain embodiments of a compound of Formula (I), each of R¹ and R² is hydrogen; each of R³ and R⁴ is C₁₋₆ alkyl; and R⁵ is methyl.

In certain embodiments of a compound of Formula (I), one of R¹ and R² is hydrogen and the other of R¹ and R² is chosen from hydrogen and C₁₋₄ alkyl; R³ is hydrogen; R⁴ is chosen from C₁₋₄ alkyl, substituted C₁₋₄ alkyl wherein the substituent group is chosen from ═O, —OR¹¹, —COOR¹¹, and —NR¹¹₂, wherein each R¹¹ is independently chosen form hydrogen and C₁₋₄ alkyl; and R⁵ is methyl. In certain embodiments of a compound of Formula (I), one of R¹ and R² is hydrogen and the other of R¹ and R² is methyl; R³ is hydrogen; R⁴ is chosen from C₁₋₄ alkyl, substituted C₁₋₄ alkyl wherein the substituent group is chosen from ═O, —OR¹¹, —COOR¹¹, and —NR¹¹₂, wherein each R¹¹ is independently chosen form hydrogen and C₁₋₄ alkyl; and R⁵ is methyl. In certain embodiments of a compound of Formula (I), each of R¹ and R² is hydrogen; R³ is hydrogen; R⁴ is chosen from C₁₋₄ alkyl, substituted C₁₋₄ alkyl wherein the substituent group is chosen from ═O, —OR¹¹, —COOR¹¹, and —NR¹¹₂, wherein each R¹¹ is independently chosen form hydrogen and C₁₋₄ alkyl; and R⁵ is methyl.

In certain embodiments of a compound of Formula (I), R³ and R⁴ together with the nitrogen to which they are bonded form a C₅₋₁₀ heterocycloalkyl ring.

In certain embodiments of a compound of Formula (I), one of R¹ and R² is hydrogen and the other of R¹ and R² is chosen from hydrogen and C₁₋₆ alkyl; R³ and R⁴ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R⁵ is methyl. In certain embodiments of a compound of Formula (I), one of R¹ and R² is hydrogen and the other of R¹ and R² is methyl; R³ and R⁴ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R⁵ is methyl. In certain embodiments of a compound of Formula (I), each of R¹ and R² is hydrogen; R³ and R⁴ together with the nitrogen to which they are bonded form a ring chosen from a C₅₋₆ heterocycloalkyl, substituted C₅₋₆ heterocycloalkyl, C₅₋₆ heteroaryl, and substituted C₅₋₆ heteroaryl ring; and R⁵ is methyl.

In certain embodiments of a compound of Formula (I), one of R¹ and R² is hydrogen and the other of R¹ and R² is chosen from hydrogen and C₁₋₆ alkyl; and R³ and R⁴ together with the nitrogen to which they are bonded form a ring chosen from morpholine, piperazine, and N-substituted piperazine.

In certain embodiments of a compound of Formula (I), one of R¹ and R² is hydrogen and the other of R¹ and R² is chosen from hydrogen and C₁₋₆ alkyl; R³ and R⁴ together with the nitrogen to which they are bonded form a ring chosen from morpholine, piperazine, and N-substituted piperazine; and R⁵ is methyl.

In certain embodiments of a compound of Formula (I), R⁵ is not methyl.

In certain embodiments of a compound of Formula (I), R¹ is hydrogen, and in certain embodiments, R² is hydrogen.

In certain embodiments of a compound of Formula (I), the compound is chosen from: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; 4-{2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; 4-{2-[(2E)-3-(methoxycarbonyl)prop-2-enoyloxy]acetylamino}butanoic acid, sodium salt; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N-[2-(dimethylamino)ethyl]carbamoyl}methyl methyl(2E)but-2ene-1,4 dioate; methyl 2-(4-methylpiperazinyl)-2-oxoethyl(2E)but-2-ene-1,4-dioate; methyl {N-[(propylamino)carbonyl]carbamoyl}methyl(2E)but-2ene-1,4-dioate; 2-(4-acetylpiperazinyl)-2-oxoethyl methyl(2E)but-2ene-1,4-dioate; {N,N-bis[2-(methylethoxy)ethyl]carbamoyl}methyl methyl(2E)but-2-ene-1,4-dioate; methyl 2-(4-benzylpiperazinyl)-2-oxoethyl(2E)but-2-ene-1,4-dioate; [N,N-bis(2-ethoxyethyl)carbamoyl]methyl methyl(2E)but-2-ene-1,4-dioate; 2-{(2S)-2-[(tert-butyl)oxycarbonyl]pyrrolidinyl}-2-oxoethyl methyl(2E)but-2ene-1,4-dioate; 1-{2-{(2E)-3-(methoxycarbonyl)prop-2-enoyloxy]acetyl}(2S)pyrrolidine-2-carboxylic acid; (N-{[tert-butyl)oxycarbonyl]methyl}) —N-methylcarbamoyl)methyl methyl(2E)but-2ene 1,4-dioate; {N-(ethoxycarbonyl)methyl}-N-methylcarbamoyl}methyl methyl(2E)but-2-ene-1,4-dioate; methyl 1-methyl-2-morpholin-4-yl-2-oxoethyl(2E)but-2-ene-1,4-dioate; [N,N-bis(2-methoxyethyl)carbamoyl]ethyl methyl(2E)but-2-ene-1,4-dioate; (N,N-dimethylcarbamoyl)ethyl methyl(2E)but-2-ene-1,4-dioate; 2-{2-[(2E)-3-(methoxy carbonyl)prop-2-enoyloxy]-N-methylacetylamino}acetic acid; (N-{[(tert-butyl)oxycarbonyl]methyl}carbamoyl)methyl methyl(2E)but-2-ene-1,4-dioate; (2E)but-methyl-N-{[(methylethyl)oxycarbonyl]methyl}carbamoyl)methyl(2E)but-2-ene-1,4-dioate; {N-[(ethoxycarbonyl)methyl]-N-benzylcarbamoyl}methyl methyl(2E)but-2-ene-1,4-dioate; {N-[(ethoxycarbonyl)methyl]-N-benzylcarbamoyl}ethyl methyl(2E)but-2-ene-1,4-dioate; {N-[(ethoxycarbonyl)methyl]-N-methylcarbamoyl}ethyl methyl(2E)but-2-ene-1,4-dioate; (1S)-1-methyl-2-morpholin-4-yl-2-oxo ethyl methyl(2E)but-2-ene-1,4-dioate; (1 S)-1-[N,N-bis(2-methoxyethyl)carbamoyl]ethyl methyl(2E)but-2-ene-1,4-dioate; (1R)-1-(N,N-diethylcarbamoyl)ethyl methyl(2E)but-2-ene-1,4-dioate; and a pharmaceutically acceptable salt of any of the foregoing.

In certain embodiments of a compound of Formula (I), the compound is chosen from: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; {2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N-[2-(dimethylamino)ethyl]carbamoyl}methyl methyl(2E)but-2ene-1,4-dioate; (N-[(methoxycarbonyl)ethyl]carbamoyl)methyl methyl(2E)but-2-en-1,4-dioate; 2-{2-[(2E)-3-(methoxycarbonyl)prop-2-enoyloxy]acetylamino}propanoic acid; and a pharmaceutically acceptable salt of any of the foregoing.

In certain embodiments of a compound of Formula (I), R³ and R⁴ are independently chosen from hydrogen, C₁₋₆ alkyl, substituted C₁₋₆ alkyl, C₆₋₁₀ aryl, substituted C₆₋₁₀ aryl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₆₋₁₀ heteroaryl, substituted C₆₋₁₀ heteroaryl, C₄₋₁₂ heterocycloalkylalkyl, substituted C₄₋₁₂ heterocycloalkylalkyl, C₇₋₁₂ heteroarylalkyl, substituted C₇₋₁₂ heteroarylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl.

In some embodiments, the compound that metabolizes to MMF is a compound of Formula II:

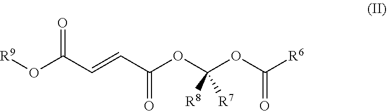

or a pharmaceutically acceptable salt thereof, wherein

R⁶ is chosen from C₁₋₆ alkyl, substituted C₁₋₆ alkyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl;
R⁷ and R⁸ are independently chosen from hydrogen, C₁₋₆ alkyl, and substituted C₁₋₆ alkyl; and
R⁹ is chosen from C₁₋₆ alkyl and substituted C₁₋₆ alkyl;
wherein each substituent group is independently chosen from halogen, —OH, —CN, —CF₃, —O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In certain embodiments of a compound of Formula (II), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl.

In certain embodiments of a compound of Formula (I), each substituent group is independently chosen from ═O, C₁₋₄ alkyl, and —COOR¹¹ wherein R¹¹ is chosen from hydrogen and C₁₋₄ alkyl.

In certain embodiments of a compound of Formula (II), one of R⁷ and R⁸ is hydrogen and the other of R⁷ and R⁸ is C₁₋₆ alkyl. In certain embodiments of a compound of Formula (II), one of R⁷ and R⁸ is hydrogen and the other of R⁷ and R⁸ is C₁₋₄ alkyl.

In certain embodiments of a compound of Formula (II), one of R⁷ and R⁸ is hydrogen and the other of R⁷ and R⁸ is chosen from methyl, ethyl, n-propyl, and isopropyl. In certain embodiments of a compound of Formula (II), each of R⁷ and R⁸ is hydrogen.

In certain embodiments of a compound of Formula (II), R⁹ is chosen from substituted C₁₋₆ alkyl and —OR¹¹ wherein R¹¹ is independently C₁₋₄ alkyl.

In certain embodiments of a compound of Formula (II), R⁹ is C₁₋₆ alkyl, in certain embodiments, R⁹ is C₁₋₃ alkyl; and in certain embodiments, R⁹ is chosen from methyl and ethyl.

In certain embodiments of a compound of Formula (II), R⁹ is methyl.

In certain embodiments of a compound of Formula (II), R⁹ is chosen from ethyl, n-propyl, isopropyl, n-butyl, sec-butyl, isobutyl, and tert-butyl.

In certain embodiments of a compound of Formula (II), R⁹ is chosen from methyl, ethyl, n-propyl, isopropyl, n-butyl, isobutyl, and tert-butyl.

In certain embodiments of a compound of Formula (II), R⁶ is C₁₋₆ alkyl; one of R⁷ and R⁸ is hydrogen and the other of R⁷ and R⁸ is C₁₋₆ alkyl; and R⁹ is chosen from C₁₋₆ alkyl and substituted C₁₋₆ alkyl.

In certain embodiments of a compound of Formula (II), R⁶ is —OR¹⁰.

In certain embodiments of a compound of Formula (II), R¹⁰ is chosen from C₁₋₄ alkyl, cyclohexyl, and phenyl.

In certain embodiments of a compound of Formula (II), R⁶ is chosen from methyl, ethyl, n-propyl, and isopropyl; one of R⁷ and R⁸ is hydrogen and the other of R⁷ and R⁸ is chosen from methyl, ethyl, n-propyl, and isopropyl.

In certain embodiments of a compound of Formula (II), R⁶ is substituted C₁₋₂ alkyl, wherein each of the one or more substituent groups are chosen from —COOH, —NHC(O)CH₂NH₂, and —NH₂.

In certain embodiments of a compound of Formula (II), R⁶ is chosen from ethoxy, methylethoxy, isopropyl, phenyl, cyclohexyl, cyclohexyloxy, —CH(NH₂CH₂COOH, —CH₂CH(NH₂)COOH, —CH(NHC(O)CH₂NH₂)—CH₂COOH, and —CH₂CH(NHC(O)CH₂NH₂)—COOH.

In certain embodiments of a compound of Formula (II), R⁹ is chosen from methyl and ethyl; one of R⁷ and R⁸ is hydrogen and the other of R⁷ and R⁸ is chosen from hydrogen, methyl, ethyl, n-propyl, and isopropyl; and R⁶ is chosen from C₁₋₃ alkyl, substituted C₁₋₂ alkyl wherein each of the one or more substituent groups are chosen —COOH, —NHC(O)CH₂NH₂, and —NH₂, —OR¹⁰ wherein R¹⁰ is chosen from C₁₋₃ alkyl and cyclohexyl, phenyl, and cyclohexyl.

In certain embodiments of a compound of Formula (II), the compound is chosen from: ethoxycarbonyloxyethyl methyl(2E)but-2-ene-1,4-dioate; methyl(methylethoxycarbonyloxy)ethyl(2E)but-2-ene-1,4-dioate; (cyclohexyloxycarbonyloxy)ethyl methyl(2E)but-2-ene-1,4-dioate; and a pharmaceutically acceptable salt of any of the foregoing.

In certain embodiments of a compound of Formula (II), the compound is chosen from: methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; methyl 2-methyl-1-phenylcarbonyloxypropyl(2E)but-2-ene-1,4-dioate; and a pharmaceutically acceptable salt of any of the foregoing.

In certain embodiments of a compound of Formula (II), the compound is chosen from: ethoxycarbonyloxyethyl methyl(2E)but-2-enc-1,4-dioate; methyl(methylethoxycarbonyloxy)ethyl(2E)but-2-ene-1,4-dioate; methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; (cyclohexyloxycarbonyloxy)ethyl methyl(2E)but-2-ene-1,4-dioate; methyl 2-methyl-1-phenylcarbonyloxypropyl(2E)but-2-ene-1,4-dioate; 3-({[(2E)-3-(methoxycarbonyl)prop-2-enoyloxy]methyl}oxycarbonyl)(3S)-3-aminopropanoic acid, 2,2,2-trifluoroacetic acid; 3-({[(2E)-3-(methoxycarbonyl)prop-2-enoyloxy]methyl}oxycarbonyl)(2S)-2-aminopropanoic acid, 2,2,2-trifluoroacetic acid; 3-({[(2E)-3-(methoxycarbonyl)prop-2-enoyloxy]methyl}oxycarbonyl)(3S)-3-(2-aminoacetylamino)propanoic acid, 2,2,2-trifluoroacetic acid; 3-({[(2E)-3-(methoxycarbonyl)prop-2-enoyloxy]methyl}oxycarbonyl)(2S)-2-aminopropanoic acid, 2,2,2-trifluoroacetic acid; 3-{[(2E)-3-(methoxycarbonyl)prop-2enoyloxy]ethoxycarbonyloxy}(2S)-2-aminopropanoic acid, chloride; and a pharmaceutically acceptable salt of any of the foregoing.

The compounds of Formulae (I)-(II) may be prepared using methods known to those skilled in the art, or the methods disclosed in U.S. Pat. No. 8,148,414 B2.

In another embodiment is provided silicon-containing compounds, which like DMF and the compounds of Formulae (I)-(II), can metabolize into MMF upon administration.

In some embodiments, the compound that metabolizes to MMF is a compound of Formula (III):

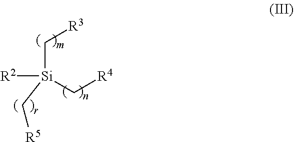

or a pharmaceutically acceptable salt thereof, wherein:
R² is C₁-C₁₀ alkyl, C₅-C₁₅ aryl, hydroxyl, —O—C₁-C₁₀ alkyl, or —O—C₅-C₁₅ aryl;
each of R³, R⁴, and R⁵, independently, is C₁-C₁₀ alkyl, C₅-C₁₅ saryl, hydroxyl, —O—C₁-C₁₀ alkyl, —O—C₅-C₁₅ aryl, or

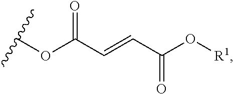

wherein R¹ is C₁-C₂₄ alkyl or C₅-C₅₀ aryl; each of which can be optionally substituted; and
each of m, n, and r, independently, is 0-4;
provided that at least one of R³, R⁴, and R⁵ is

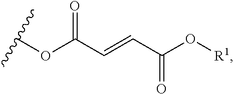

Another group of compounds of Formula III include compounds wherein R¹ is optionally substituted C₁-C₂₄ alkyl. Another group of compounds of Formula III include compounds wherein R¹ is optionally substituted C₁-C₆ alkyl. Another group of compounds of Formula III include compounds wherein R¹ is optionally substituted methyl, ethyl, or isopropyl. Another group of compounds of Formula III include compounds wherein R¹ is optionally substituted C₅-C₅₀ aryl. Another group of compounds of Formula III include compounds wherein R¹ is optionally substituted C₅-C₁₀ aryl. Another group of compounds of Formula III include compounds wherein R² is C₁-C₁₀ alkyl. Another group of compounds of Formula III include compounds wherein R² is optionally substituted C₁-C₆ alkyl. Another group of compounds of Formula III include compounds wherein R² is optionally substituted methyl, ethyl, or isopropyl. Another group of compounds of Formula III include compounds wherein R² is optionally substituted C₅-C₁₅ aryl. Another group of compounds of Formula III include compounds wherein R² is optionally substituted C₅-C₁₀ aryl.

In a further embodiment, the compound that metabolizes to MMF is a compound of Formula (III):

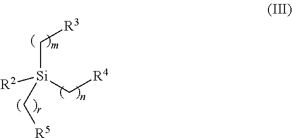

or a pharmaceutically acceptable salt thereof, wherein
R² is C₁-C₁₀ alkyl, C₆-C₁₀ aryl, hydroxyl, —O—C₁-C₁₀ alkyl, or —O—C₆-C₁₀ aryl; each of R³, R⁴, and R⁵, independently, is C₁-C₁₀ alkyl, C₆-C₁₀ aryl, hydroxyl, —O—C₁-C₁₀ alkyl, —O—C₆-C₁₀ aryl, or

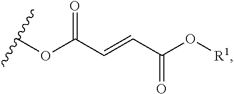

wherein R¹ is C₁-C₂₄ alkyl or C₆-C₁₀ aryl; each of which can be optionally substituted; and
each of m, n, and r, independently, is 0-4;
provided that at least one of R³, R⁴, and R⁵ is

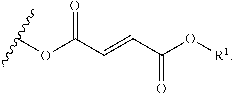

In some embodiments, the compound that metabolizes to MMF is chosen from (dimethylsilanediyl)dimethyl difumarate; methyl ((trimethoxysilyl)methyl) fumarate; methyl ((trihydroxysilyl)methyl) fumarate; trimethyl (methylsilanetriyl) trifumarate; and a pharmaceutically acceptable salt of any of the foregoing.

In some embodiments, the compound that metabolizes to MMF is a compound of Formula (IV):

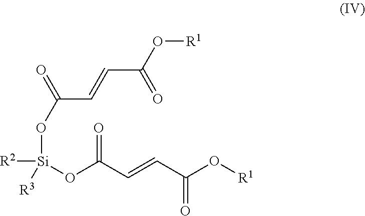

or a pharmaceutically acceptable salt thereof, wherein:
each of, independently, R² and R³, is C₁-C₁₀ alkyl or C₅-C₁₅ aryl.
R² and R³ can be the same or different, can be optionally substituted, and independently can be selected from the group consisting of C₁-C₁₀ alkyl or C₅-C₁₅ aryl.

In another embodiment, compounds of Formula IV include compounds wherein R¹ is optionally substituted C₁-C₂₄ alkyl. Another group of compounds of Formula IV include compounds wherein R¹ is optionally substituted C₁-C₆ alkyl. Another group of compounds of Formula IV include compounds wherein R¹ is optionally substituted methyl, ethyl, or isopropyl. Another group of compounds of Formula IV include compounds wherein R¹ is optionally substituted C₅-C₅₀ aryl. Another group of compounds of Formula IV include compounds wherein R¹ is optionally substituted C₅-C₁₀ aryl. Another group of compounds of Formula IV include compounds wherein each of R² and R³ is, independently, optionally substituted C₁-C₁₀ alkyl. Another group of compounds of Formula IV include compounds wherein each of R² and R³ is, independently, optionally substituted C₁-C₆ alkyl. Another group of compounds of Formula IV include compounds wherein each of R² and R³ is, independently, optionally substituted methyl, ethyl, or isopropyl. Another group of compounds of Formula IV include compounds wherein each of R² and R³ is, independently, optionally substituted C₅-C₁₅ aryl. Another group of compounds of Formula IV include compounds wherein each of R² and R³ is, independently, optionally substituted C₅-C₁₀ aryl.

In a further embodiment, the compound that metabolizes to MMF is a compound of Formula (IV):

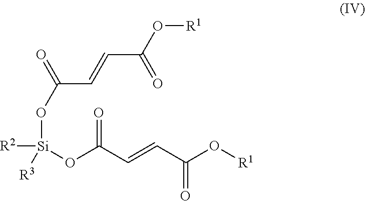

or a pharmaceutically acceptable salt thereof, wherein:
R¹ is C₁-C₂₄ alkyl or C₆-C₁₀ aryl; and
each of, independently, R² and R³, is C₁-C₁₀ alkyl or C₆-C₁₀ aryl.

In some embodiments, the compound that metabolizes to MMF is a compound of Formula (V):

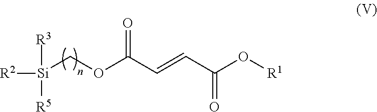

or a pharmaceutically acceptable salt thereof, wherein:
R¹ is C₁-C₂₄ alkyl or C₅-C₅₀ aryl;
each of R², R³, and R⁵, independently, is hydroxyl, C₁-C₁₀ alkyl, C₅-C₁₅ aryl, —O—C₁-C₁₀ alkyl, or —O—C₅-C₁₅ aryl; and
n is 1 or 2.

In another embodiment, compounds of Formula V include compounds wherein R¹ is optionally substituted C₁-C₂₄ alkyl. Another group of compounds of Formula V include compounds wherein R¹ is optionally substituted C₁-C₆ alkyl. Another group of compounds of Formula V include compounds wherein R¹ is optionally substituted methyl, ethyl, or isopropyl. Another group of compounds of Formula V include compounds wherein R¹ is optionally substituted C₅-C₅₀ aryl. Another group of compounds of Formula V include compounds wherein R¹ is optionally substituted C₅-C₁₀ aryl. Another group of compounds of Formula V include compounds wherein each of R², R³, and R⁵ is, independently, hydroxyl. Another group of compounds of Formula V include compounds wherein each of R², R³, and R⁵ is, independently, optionally substituted C₁-C₁₀ alkyl. Another group of compounds of Formula V include compounds wherein each of R², R³, and R⁵ is, independently, optionally substituted C₁-C₆ alkyl. Another group of compounds of Formula V include compounds wherein each of R², R³, and R⁵ is, independently, optionally substituted methyl, ethyl, or isopropyl. Another group of compounds of Formula V include compounds wherein each of R², R³, and R⁵ is, independently, optionally substituted C₅-C₁₅ aryl. Another group of compounds of Formula V include compounds wherein each of R², R⁶, and R⁵ is, independently, optionally substituted C₅-C₁₀ aryl.

In a further embodiment, the compound that metabolizes to MMF is a compound of Formula (V):

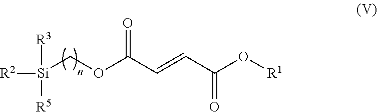

or a pharmaceutically acceptable salt thereof, wherein:
R¹ is C₁-C₂₄ alkyl or C₆-C₁₀ aryl;
each of R², R³, and R⁵, independently, is hydroxyl, C₁-C₁₀ alkyl, C₆-C₁₀ aryl, —O—C₁-C₁₀ alkyl, or —O—C₆-C₁₀ aryl; and
n is 1 or 2.

In some embodiments, the compound that metabolizes to MMF is a compound of Formula (VI):

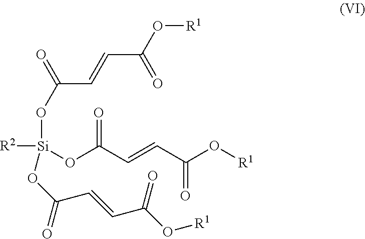

or a pharmaceutically acceptable salt thereof, wherein:

R¹ is C₁-C₂₄ alkyl or C₅-C₅₀ aryl; and

R² is C₁-C₁₀ alkyl.

In another embodiment, compounds of Formula VI include compounds wherein R¹ is optionally substituted C₁-C₂₄ alkyl. Another group of compounds of Formula VI include compounds wherein R¹ is optionally substituted C₁-C₆ alkyl. Another group of compounds of Formula VI include compounds wherein R¹ is optionally substituted methyl, ethyl, or isopropyl. Another group of compounds of Formula VI include compounds wherein R¹ is optionally substituted C₅-C₅₀ aryl. Another group of compounds of Formula VI include compounds wherein R¹ is optionally substituted C₅-C₁₀ aryl. Another group of compounds of Formula VI include compounds wherein R² is optionally substituted C₁-C₆ alkyl. Another group of compounds of Formula VI include compounds wherein R² is optionally substituted methyl, ethyl, or isopropyl.

In a further embodiment, the compound that metabolizes to MMF is a compound of Formula (VI):

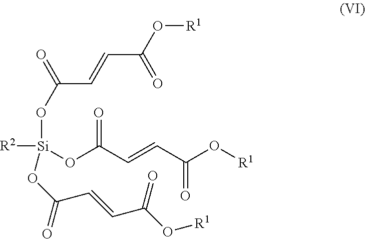

or a pharmaceutically acceptable salt thereof, wherein:
R¹ is C₁-C₂₄ alkyl or C₆-C₁₀ aryl; and
R² is C₁-C₁₀ alkyl.

The compounds of Formulae (III)-(VI) may be prepared using methods known to those skilled in the art, or the methods disclosed in the present invention.

Specifically, the compounds of this invention of Formula IV may be prepared by the exemplary reaction in Scheme 1.

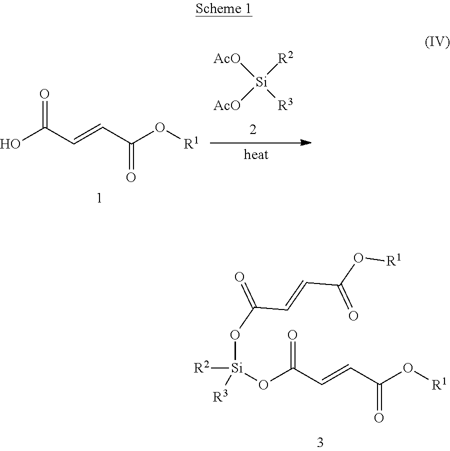

wherein R¹, R², and R³ are each defined above for Formula IV.

Reaction of fumaric acid ester 1 with silane diacetate intermediate 2 in a refluxing organic solvent such as diethyl ether, toluene, or hexane to give the desired siloxane 3.

Some of the fumaric acid esters 1 are commercially available. Fumaric acid ester 1 can also be prepared, for example, using synthetic methods known by one of ordinary skill in the art. For example, fumaric acid can be converted by reacting alcohol (R¹—OH) with a catalytic amount of p-toluene sulfonic acid at room temperature for a few hours to overnight as shown in Scheme 2.

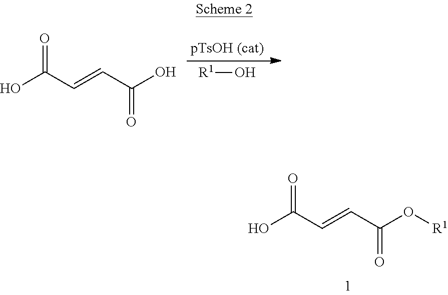

wherein R¹ is defined above for Formula III.

Alternatively, fumaric acid ester 1 can be prepared by reacting alcohol (R¹—OH) under the coupling conditions of hydroxybenzotriazole (HOBT), 1-ethyl-3-(3-dimethylaminopropyl)carbodiimide (EDCI), and diisopropyl amine (DIPEA) as shown in Scheme 3.

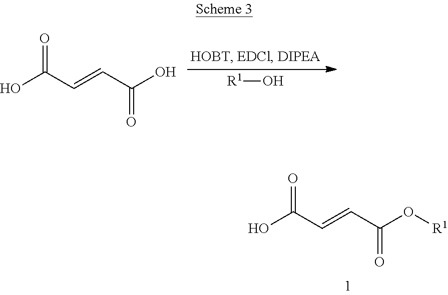

wherein R¹ is defined above for Formula III.

Some of the silanes that can be used in the present invention are commercially available. Commercially available silyl halides include trimethylsilyl chloride, dichloro-methylphenylsilane, dimethyldichlorosilane, methyltrichlorosilane, (4-aminobutyl)diethoxymethylsilane, trichloro(chloromethyl)silane, trichloro(dichlorophenyl)silane, trichloroethylsilane, trichlorophenylsilane, and trimethylchlorosilane. Commercial sources for silyl halides include Sigma Aldrich and Acros Organics.

Silanes used in the present invention can be prepared, for example, using synthetic methods known by one of ordinary skill in the art. For example, trichlorosilane may be prepared by the exemplary reaction in Scheme 4.

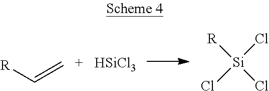

The silylation of styrene derivatives catalyzed by palladium is described in Zhang, F. and Fan, Q.-H., Organic & Biomolecular Chemistry 7:4470-4474 (2009) and in Bell, J. R., et al., Tetrahedron 65:9368-9372 (2009).

Diacetate intermediate 2 may be prepared by treatment of dichlorosubstituted silicon compound 4 with sodium acetate in diethyl ether under reflux as shown in Scheme 5.

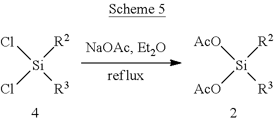

wherein R² and R³ are each defined above for Formula IV.

Specifically, the compounds of this invention of Formula V may be prepared by the exemplary reaction in Scheme 6.

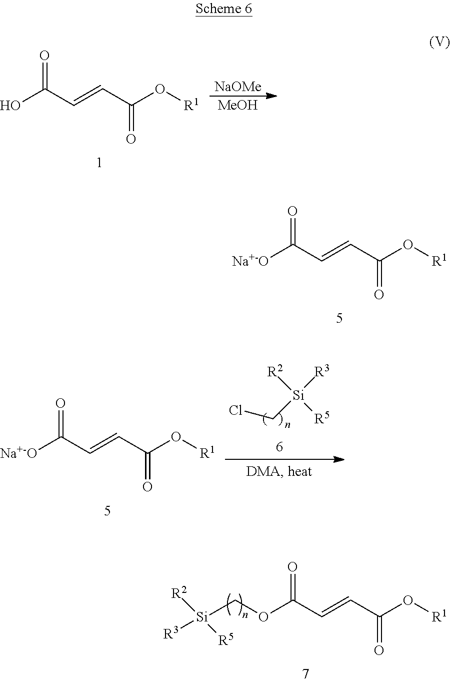

wherein R¹, R², R³, and R⁵ are as defined above for Formula V.

Fumaric acid ester 1 can be converted to the sodium salt 5 using, for example, sodium methoxide in methanol at room temperature. Removal of the solvent would afford sodium salt 5. Treatment of the sodium salt 5 with silane 6 in an organic solvent such as dimethylformamide under reflux would generate ester 7. The synthesis of structurally related (trimethoxysilyl)-methyl esters is described in Voronkov, M. G., et al., Zhurnal Obshchei Khimii 52:2052-2055 (1982).

Alternatively, the compounds of this invention of Formula V may be prepared by the exemplary reaction in Scheme 7.

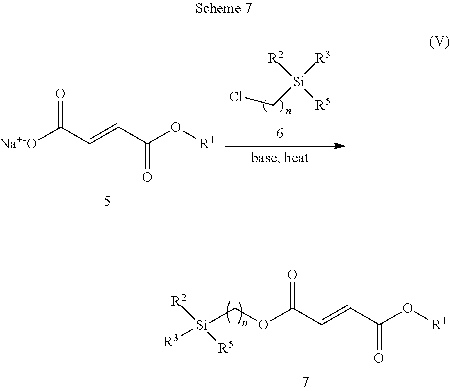

wherein R¹, R⁴, R⁵, R⁶, and n are as defined above for Formula V.

Treatment of the sodium salt 5 with silane 6 in an organic solvent such as dimethylformamide under heating with or without an acid scavenger would generate ester 7.

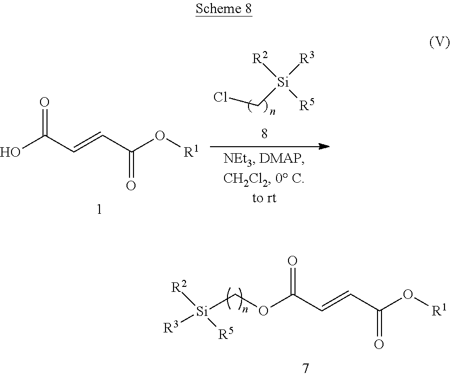

wherein R¹, R⁴, R⁵, R⁶, and n are as defined above for Formula V.

Reaction of fumaric acid ester 1 with tri-substituted silane alcohol 8 in methylene chloride with mild base such as triethyl amine and 4-N,N-dimethyl amino pyridine (DMAP) at room temperature generates fumarate 7. See Coelho, P. J., et al., Eur. J. Org. Chem. 3039-3046 (2000).

Specifically, the compounds of this invention of Formula VI can be prepared by the exemplary reaction in Scheme 9.

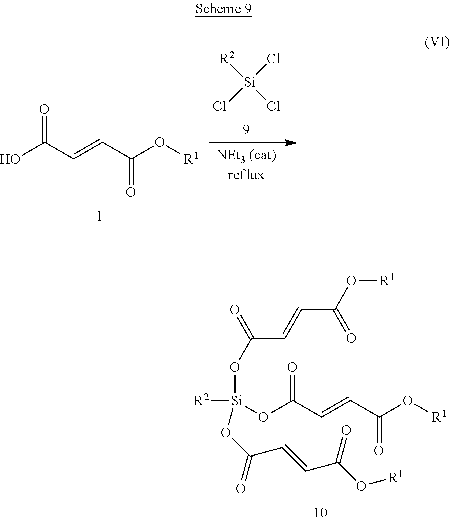

wherein R¹ and R² are as defined above for Formula VI.

Reaction of fumaric acid 1 with trichlorosilane 9 in a refluxing organic solvent such as hexane or toluene using a catalytic amount of a base such as triethylamine generates the trifumarate silane 10. The reaction of acetic and methacrylic acids with 1-silyladamantanes is described in Fedotov, N. S., et al., Zhurnal Obshchei Khimii 52:1837-1842 (1982).

The compounds and pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The amount of active ingredient that can be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient can also depend upon the therapeutic or prophylactic agent, if any, with which the ingredient is co-administered.

In some embodiments, the compounds and pharmaceutical compositions of the invention can be administered in an amount ranging from about 1 mg/kg to about 50 mg/kg (e.g., from about 2.5 mg/kg to about 20 mg/kg or from about 2.5 mg/kg to about 15 mg/kg). The amount of the compounds and pharmaceutical compositions of the invention administered will also vary, as recognized by those skilled in the art, dependent on route of administration, excipient usage, and the possibility of co-usage with other therapeutic treatments including use of other therapeutic agents.

For example, the compounds and pharmaceutical compositions of the invention can be administered to a subject, for example orally, in an amount of from about 0.1 g to about 1 g per day, or for example, in an amount of from about 100 mg to about 800 mg per day.

The amount of compounds and pharmaceutical compositions of the invention may be administered once a day or in separate administrations of 2, 3, 4, 5 or 6 equal doses per day.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which may be used pharmaceutically. For example, the preparations, particularly those preparations which may be administered orally and which may be used for the preferred type of administration, such as tablets, dragees, and capsules, and also preparations which may be administered rectally, such as suppositories, as well as suitable solutions for administration by injection or orally, contain from about 0.01 to 99 percent, preferably from about 0.25 to 75 percent of active compound(s), together with the excipient.

Also included within the scope of the present invention are the non-toxic pharmaceutically acceptable salts of the compounds of the present invention. Acid addition salts are formed by mixing a solution of a compound that metabolizes to MMF with a solution of a pharmaceutically acceptable non-toxic acid such as hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, gluconate, glucaronate, saccharate, formate, benzoate, glutamate, methanesulfonate, ethanesulfonate, benzenesulfonate, p-toluenesulfonate, and pamoate. Acceptable base salts include aluminum, calcium, lithium, magnesium, potassium, sodium, zinc, and diethanolamine salts.

The pharmaceutical compositions of the invention may be administered to any animal which may experience the beneficial effects of the compounds of the invention. Foremost among such animals are mammals, e.g., humans and veterinary animals, although the invention is not intended to be so limited.

The pharmaceutical preparations of the present invention are manufactured in a manner which is itself known, for example, by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments may be added to the tablets or dragee coatings, for example, for identification or in order to characterize combinations of active compound doses.

In one embodiment, the pharmaceutical preparations comprise a capsule containing the compound or pharmaceutical composition of the present invention in the form of an enteric-coated microtablet. The coating of the microtablet may be composed of different layers. The first layer may be a methacrylic acid-methyl methacrylate copolymer/isopropyl solution which isolates the tablet cores from potential hydrolysis from the next applied water suspensions. The enteric coating of the tablet may then be conferred by an aqueous methacrylic acid-ethyl acrylate copolymer suspension.

When the compound that metabolizes to MMF is administered to a human, the compound quickly metabolizes to MMF. The pharmacokinetics properties (e.g., Cₘₐₓ and AUC) are therefore measured based on the concentration of MMF in the plasma after administration. The pharmacokinetics properties can be determined after single dosing or at steady state. In some embodiments, patients orally administered a dosage form described above containing a compound that metabolizes to MMF exhibit a time to maximum plasma MMF concentration (Tₘₐₓ) of, for example, from about 1.5 hours to about 3.5 hours, from about 1.75 hours to about 3.25 hours, or from about 2 hours to about 2.5 hours.

In some embodiments, patients orally administered a dosage form described above containing a compound that metabolizes to MMF exhibit a mean MMF plasma area under the curve 0-12 (AUC₀₋₁₂) of about 2.36 h·mg/L to about 5.50 h·mg/L, from about 2.75 h·mg/L to about 5.10 h·mg/L, or from about 3.14 h·mg/L to about 4.91 h·mg/L. In one embodiment, patients exhibit a mean AUC₀₋₁₂ of about 3.93 h·mg/L.

In some embodiments, patients orally administered a dosage form described above containing a compound that metabolizes to MMF exhibit a mean MMF plasma area under the curve 0-infinity (AUC_{0-infinity}) of about 2.4 h·mg/L to about 5.6 h·mg/L, from about 2.75 h·mg/L to about 5.10 h·mg/L, or from about 3.14 h·mg/L to about 4.91 h·mg/L. In one embodiment, patients exhibit a mean AUC_{0-infinity} of about 3.93 h·mg/L.

In some embodiments patients orally administered a dosage form described above containing a compound that metabolizes to MMF twice daily exhibit a mean MMF plasma overall area under the curve (AUCₒᵥₑᵣₐₗₗ) of about 4.81 h·mg/mL to about 11.2 h·mg/mL, or from about 6.40 h·mg/L to about 10.1 h·mg/L. In one embodiment, patients exhibit a mean AUCₒᵥₑᵣₐₗₗ of about 8.02 h·mg/L when orally administered the dosage forms twice daily.

In some embodiments, patients orally administered a dosage form described above containing a compound that metabolizes to MMF exhibit a mean MMF plasma concentration (Cₘₐₓ) of from about 1.45 mg/L to about 3.39 mg/L, from about 1.69 mg/L to about 3.15 mg/L, or from about 1.93 mg/L to about 3.03 mg/L. In one embodiment, patients exhibit a mean Cₘₐₓ of about 2.42 mg/L.

In one embodiment, patients orally administered a dosage form described above containing a compound that metabolizes to MMF twice daily exhibit a mean Cₘₐₓ of about 1.02 mg/L, to about 2.41 mg/L, or about 1.37 mg/L to about 2.15 mg/L. In one embodiment, patients exhibit a mean Cₘₐₓ of about 1.72 mg/L when orally administered the dosage forms twice daily.

In another embodiment is provided a composition comprising dimethyl fumarate and one or more excipients, wherein a total amount of dimethyl fumarate in the composition ranges, for example, from about 43% w/w to about 95% w/w, based on the total weight of the composition, excluding the weight of any coating.

The total amount of dimethyl fumarate in the composition can range, for example, from about 43% w/w to about 95% w/w, from about 50% w/w to about 95% w/w, from about 50% w/w to about 85% w/w, from about 55% w/w to about 80% w/w, from about 60% w/w to about 75% w/w, from about 60% w/w to about 70% w/w, or from about 65% w/w to about 70% w/w, based on the total weight of the composition, excluding the weight of any coating.

The composition can comprise dimethyl fumarate, for example, in about 43% w/w, about 45% w/w, about 50% w/w, about 55% w/w, about 60% w/w, about 65% w/w, about 70% w/w, about 75% w/w, about 80% w/w, about 90% w/w, or about 95% w/w, based on the weight of the composition, excluding the weight of any coating. For example, the composition can contain about 65% to about 95% w/w (e.g., 65% w/w) of DMF.

Some or all of the dimethyl fumarate in the composition can have a particle size of 250 microns or less. For example, and without being limiting, at least 80%, at least 90%, at least 95%, at least 97%, or at least 99% of the dimethyl fumarate in the composition can have a particle size of 250 microns or less. Particle size can be measured, for example, by sieve analysis, air elutriation analysis, photoanalysis, electrical counting methods, electroresistance counting methods, sedimentation techniques, laser diffraction methods, acoustic spectroscopy, or ultrasound attenuation spectroscopy. In one embodiment, the particle size is measured using laser diffraction methods.

The composition can comprise a total amount of excipient(s), for example, in an amount of about 5.0% w/w to about 57% w/w, based on the total weight of the composition, excluding the weight of any coating.

The composition can comprise a total amount of excipient(s) in an amount ranging, for example, from about 5% w/w to about 57% w/w, from about 15% w/w to about 57% w/w, from about 20% w/w to about 57% w/w, from about 25% w/w to about 57% w/w, from about 30% w/w to about 57% w/w, from about 35% w/w to about 57% w/w, from about 40% to about 57% w/w, from about 45% w/w to about 57% w/w, from about 50% w/w to about 57% w/w, from about 55% w/w to about 57% w/w, from about 5% w/w to about 55% w/w, from about 5% w/w to about 50% w/w, from about 5% w/w to about 45% w/w, from about 5% w/w to about 40% w/w, from about 5% w/w to about 35% w/w, from about 5% w/w to about 30% w/w, from about 5% w/w to about 25% w/w, from about 5% w/w to about 20% w/w, from about 5% w/w to about 15% w/w, from about 15% w/w to about 55% w/w, from about 20% w/w to about 50% w/w, from about 25% w/w to about 45% w/w, from about 30% w/w to about 40% w/w, from about 35% to about 40% w/w, based on the total weight of the composition, excluding the weight of any coating.

The excipient(s) can be, for example, one or more selected from the group consisting of a filler (or a binder), a glidant, a disintegrant, a lubricant, or any combination thereof.

The number of excipients that can be included in a composition is not limited.

Examples of fillers or binders include, but are not limited to, ammonium alginate, calcium carbonate, calcium phosphate, calcium sulfate, cellulose, cellulose acetate, compressible sugar, confectioner's sugar, dextrates, dextrin, dextrose, erythritol, ethylcellulose, fructose, glyceryl palmitostearate, hydrogenated vegetable oil type I, isomalt, kaolin, lactitol, lactose, mannitol, magnesium carbonate, magnesium oxide, maltodextrin, maltose, mannitol, medium chain triglycerides, microcrystalline cellulose, polydextrose, polymethacrylates, simethicone, sodium alginate, sodium chloride, sorbitol, starch, sucrose, sugar spheres, sulfobutylether beta-cyclodextrin, talc, tragacanth, trehalsoe, polysorbate 80, and xylitol. In one embodiment, the filler is microcrystalline cellulose. The microcrystalline cellulose can be, for example, PROSOLV SMCC® 50, PROSOLV SMCC® 90, PROSOLV SMCC® HD90, PROSOLV SMCC® 90 LM, and any combination thereof.

Examples of disintegrants include, but are not limited to, hydroxypropyl starch, alginic acid, calcium alginate, carboxymethylcellulose calcium, carboxymethylcellulose sodium, powdered cellulose, chitosan, colloidal silicon dioxide, croscarmellose sodium, crospovidone, docusate sodium, guar gum, hydroxypropyl cellulose, low substituted hydroxypropyl cellulose, magnesium aluminum silicate, methylcellulose, microcrystalline cellulose, polacrilin potassium, povidone, sodium alginate, sodium starch glycolate, starch, and pregelatinized starch. In one embodiment, the disintegrant is croscarmellose sodium.

Examples of glidants include, but are not limited to, calcium phosphate, calcium silicate, powdered cellulose, magnesium silicate, magnesium trisilicate, silicon dioxide, talcum and colloidal silica, and colloidal silica anhydrous. In one embodiment, the glidant is colloidal silica anhydrous, talc, or a combination thereof.

Examples of lubricants include, but are not limited to, canola oil, hydroxyethyl cellulose, lauric acid, leucine, mineral oil, poloxamers, polyvinyl alcohol, talc, octyldodecanol, sodium hyaluronate, sterilizable maize starch, triethanolamine, calcium stearate, magnesium stearate, glycerin monostearate, glyceryl behenate, glyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, potassium benzoate, sodium benzoate, sodium chloride, sodium lauryl sulfate, stearic acid, talc, and zinc stearate. In one embodiment, the lubricant is magnesium stearate.

The composition can comprise a total amount of filler(s) in an amount ranging from about 3.5% w/w to about 55% w/w of the composition, based on the total weight of the composition, excluding the weight of any coating.

The filler(s) can be comprised in the composition, for example, in a total amount, for example, ranging from about 5% w/w to about 55% w/w, from about 10% w/w to about 55% w/w, from about 15% w/w to about 55% w/w, from about 20% w/w to about 55% w/w, from about 25% w/w to about 55% w/w, from about 30% w/w to about 55% w/w, from about 35% w/w to about 55% w/w, from about 40% w/w to about 55% w/w, from about 3.5% w/w to about 55% w/w, from about 3.5% to about 50%, from about 3.5% w/w to about 40% w/w, from about 3.5% w/w to about 30% w/w, from about 3.5% w/w to about 25% w/w, from about 3.5% w/w to about 20% w/w, from about 3.5% w/w to about 15% w/w, from about 15% w/w to about 40% w/w, from about 20% w/w to about 35% w/w, or from about 25% w/w to about 30% w/w, based on the total weight of the composition, excluding the weight of any coating.

The filler(s) can be comprised in the composition, for example, in a total amount of about 5% w/w, about 7% w/w, about 10% w/w, about 12% w/w, about 14% w/w, about 16% w/w, about 18% w/w, about 20% w/w, about 22% w/w, about 24% w/w, about 26% w/w, about 28% w/w, about 30% w/w, about 32% w/w, about 34% w/w, about 36% w/w, about 38% w/w, about 40% w/w, about 42% w/w, about 44% w/w, about 46% w/w, about 48% w/w, about 50% w/w, about 52% w/w, about 54% w/w, or about 55% w/w, based on the total weight of the composition, excluding the weight of any coating.

The composition can comprise a total amount of disintegrant(s), for example, in an amount ranging from about 0.2% w/w to about 20% w/w, based on the total weight of the composition, excluding the weight of any coating.

The disintegrant(s) can be contained in the composition, for example, in a total amount ranging from about 0.2% w/w to about 19% w/w, about 0.2% w/w to about 15% w/w, about 0.2% w/w to about 12% w/w, about 0.2% w/w to about 6% w/w, about 0.2% w/w to about 5% w/w, about 0.2% w/w to about 4% w/w, about 0.2% w/w to about 3% w/w, about 0.2% w/w to about 2% w/w, about 0.2% w/w to about 20% w/w, about 3% w/w to about 20% w/w, about 4% w/w to about 20% w/w, about 5% w/w to about 20% w/w, about 6% w/w to about 20% w/w, about 7% w/w to about 20% w/w, about 8% w/w to about 20% w/w, about 9% w/w to about 20% w/w, about 2% w/w to about 20% w/w, or about 3% w/w to about 20% w/w, based on the weight of the composition, excluding the weight of any-coating.

The disintegrant(s) can be contained in the composition, for example, in a total amount of about 1% w/w, about 2% w/w, about 3% w/w, about 4% w/w, about 5% w/w, about 6% w/w, about 7% w/w, about 8% w/w, about 9% w/w, about 10% w/w, about 12% w/w, about 14% w/w, about 16% w/w, about 18% w/w, or about 19% w/w, based on the total weight of the composition, excluding the weight of any coating.

The glidant(s) can be contained in the composition, for example, in a total amount ranging from about 0.1% w/w to about 9.0% w/w, based on the total weight of the composition, excluding the weight of any coating.

The glidant(s) can be contained in the composition, for example, in a total amount ranging from about 0.1% w/w to about 9.0% w/w, from about 0.1% w/w to about 8% w/w, from about 0.1% w/w to about 6% w/w, from about 0.1% w/w to about 4% w/w, from about 0.1% w/w to about 2.8% w/w, from about 0.1% w/w to about 2.6% w/w, from about 0.1% w/w to about 2.4% w/w, from about 0.1% w/w to about 2.2% w/w, from about 0.1% w/w to about 2.0% w/w, from about 0.1% w/w to about 1.8% w/w, from about 0.1% w/w to about 1.6% w/w, from about 0.1% to about 1.4% w/w, from about 0.1% w/w to about 1.2% w/w, from about 0.1% w/w to about 1.0% w/w, from about 0.1% w/w to about 0.8% w/w, from about 0.1% w/w to about 0.4% w/w, from about 0.2% w/w to about 3.0% w/w, from about 0.4% w/w to about 3.0% w/w, from about 0.6% w/w to about 3.0% w/w, from about 0.8% w/w to about 3.0% w/w, from about 1.0% w/w to about 3.0% w/w, from about 1.2% w/w to about 9.0% w/w, from about 1.4% w/w to about 9.0% w/w, from about 1.6% w/w to about 9.0%, from about 1.8% w/w to about 9.0% w/w, from about 2.0% w/w to about 9.0% w/w, from about 2.2% w/w to about 9.0% w/w, from about 2.4% w/w to about 9.0% w/w, from about 2.6% w/w to about 9.0% w/w, from about 2.8% w/w to about 9.0% w/w, from about 3.0% w/w to about 9.0% w/w, from about 4.0% w/w to about 9.0% w/w, from about 5.0% w/w to about 9.0% w/w, from about 6.0% w/w to about 9.0% w/w, from about 7.0% w/w to about 9.0% w/w, from about 8.0% w/w to about 9.0% w/w, from about 0.5% w/w to about 2.5% w/w, or from about 1.0% w/w to about 2.0% w/w, based on the total weight of the composition, excluding the weight of any coating.

The glidant(s) can be contained in the composition, for example, in a total amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.2% w/w, about 1.4% w/w, about 1.6% w/w, about 1.8% w/w, about 2.0% w/w, about 2.2% w/w, about 2.4% w/w, about 2.6% w/w, about 2.8% w/w, about 3% w/w, about 4% w/w, about 5% w/w, about 6% w/w, about 7% w/w, about 8% w/w, or about 9% w/w, based on the total weight of the composition, excluding the weight of any coating.

The lubricant(s) can be contained in the composition, for example, in a total amount ranging from about 0.1% w/w to about 3.0% w/w, based on the total weight of the composition, excluding the weight of any coating.

The lubricant(s) can be contained in the composition, for example, in a total amount ranging from about 0.1% w/w to about 2% w/w, about 0.1% w/w to about 1% w/w, from about 0.1% w/w to about 0.7% w/w, from about 0.1% w/w to about 0.6% w/w, from about 0.1% w/w to about 0.5% w/w, from about 0.1% w/w to about 0.4% w/w, from about 0.1% w/w to about 0.3% w/w, from about 0.1% w/w to about 0.2% w/w, from about 0.2% w/w to about 3.0% w/w, from about 0.3% w/w to about 3.0% w/w, from about 0.4% w/w to about 3.0% w/w, from about 0.5% w/w to about 3.0% w/w, from about 0.6% w/w to about 3.0% w/w, from about 0.7% w/w to about 3.0/w/w, from about 0.8% w/w to about 3.0% w/w, from about 0.9% w/w to about 3.0% w/w, from about 1% w/w to about 3.0% w/w, from about 2% w/w to about 3% w/w, from about 0.2% w/w to about 0.7% w/w, from about 0.3% w/w to about 0.6% w/w, or from about 0.4% w/w to about 0.5% w/w, based on the total weight of the composition, excluding the weight of any coating.

The lubricant(s) can be contained in the composition, for example, in a total amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 2.0% w/w, or about 3.0% w/w, based on the total weight of composition, excluding the weight of any coating.

In some embodiments, for example, the composition comprises one or more fillers in a total amount ranging from about 3.5% w/w to about 55% w/w, one or more disintegrants in a total amount ranging from about 0.2% w/w to about 20% w/w, one or more glidants in a total amount ranging from about 0.1% w/w to about 9.0% w/w, and one or more lubricants in a total amount ranging from about 0.1% w/w to about 3.0% w/w.

In some embodiments, for example, the composition comprises a filler, a disintegrant, a glidant, and a lubricant. In some embodiments, the filler is microcrystalline cellulose, the disintegrant is croscarmellose sodium, the glidant is colloidal silica anhydrous, and the lubricant is magnesium stearate. In other embodiments, the filler is microcrystalline cellulose, the disintegrant is croscarmellose sodium, the glidant is a combination of colloidal silica anhydrous and talc, and the lubricant is magnesium stearate.

The ingredients in the composition can be, for example, homogeneous or heterogeneously mixed. The composition ingredients can be, for example, mixed by any known method including shaking, stirring, mixing with forced air, mixing in a spinning container, and the like. The composition ingredients can be, for example, mixed all at once, or with progressive addition of one or more ingredients. The composition ingredients can be mixed in any order, for example, individually, in groups, or as a blend of all of the ingredients. For example, the glidant(s) can be mixed with the DMF and/or disintegrant(s) prior to mixing with any or all of the filler(s) and/or lubricants. The blend can also be prepared by mixing DMF, disintegrant(s) (e.g., croscarmellose sodium) and a portion of binder (e.g., microcrystalline cellulose) before then passing through a screen or sieve. The remaining binder can be mixed with lubricant(s) (e.g., magnesium stearate) before passing through a screen or sieve. These two mixtures can then be combined and mixed before adding glidant(s) (e.g., silica colloidal anhydrous). The glidant(s) can also be added to one or both of the aforementioned mixtures before they are combined and mixed to produce the final blend.

The composition can have a flowability index, for example, ranging from about 8 mm to about 24 mm. For example, the flowability index can range from about 12 mm to about 22 mm, from about 12 mm to about 20 mm, from about 12 mm to about 18 mm, from about 12 mm to about 16 mm, from about 12 mm to about 14 mm, from about 14 mm to about 24 mm, from about 16 mm to about 24 mm, from about 18 mm to about 24 mm, from about 20 mm to about 24 mm, from about 22 mm to about 24 mm, from about 14 mm to about 22 mm, or from about 16 mm to about 20 mm.

The flowability index can be, for example, less than 18 mm (e.g., about 8 mm, about 12 mm, about 14 mm, about 16 mm) with an amount of glidant(s) ranging from about 0.1% w/w to about 2.0% w/w (e.g., 1.0% w/w).

The flowability index can be measured, for example, on a FLODEX device (manufactured by Hanson Research). The following protocol, for example, can be employed: A powder sample (e.g., 50 g) is loaded into the cylinder on the FLODEX device such that the powder is within about 1 cm from the top of the cylinder. A minimum of 30 seconds is allowed to pass before testing commences. Starting with a 16 mm flow disk, the release lever is slowly turned until the closure drops open without vibration. The test is positive when the open hole at the bottom is visible when looking down from the top. If a positive result is obtained, the test is repeated with smaller and smaller disk holes until the test is negative. For negative results, the size of the flow disk hole is increased until the test is positive. The flowability index is the diameter of the smallest hole through which the sample will pass for three successive tests.

The composition can have, for example, a compressibility index ranging from about 15% to about 28%. The compressibility index can range, for example, from 17% to about 28%, from about 19% to about 28%, from about 21% to about 28%, from about 23% to about 28%, from about 25% to about 28%, from about 15% to about 26%, from about 15% to about 24%, from about 15% to about 22%, from about 15% to about 20%, from about 15% to about 18%, from about 17% to about 26%, from about 19% to about 24%, or from about 20% to about 22%.

The composition can have a compressibility index, for example, of about 16%, about 17%, about 18%, about 19%, about 20%, about 21%, about 22%, about 23%, about 24%, about 25%, about 26%, or about 27%.

The compressibility index can be defined, for example, by the formula: (((Vₒ−V_{f})/Vₒ)×100%) where Vₒ is unsettled apparent volume of the particle and V_{f} is the final tapped volume of the powder. The compressibility index can be determined, for example, as follows: powder is placed in a container and the powder's unsettled apparent volume (Vₒ) is noted. Next, the powder is tapped until no further volume changes occur. At this point, the final tapped volume of the powder is measured (V_{f}). The compressibility index is then calculated by using the formula above.

In some embodiments, the composition can be in the form of a powder (not compressed) or a compact (compressed). The shape of the compact is not limited and can be, for example, cubic, spherical, or cylindrical (e.g., disc-shaped).

The compact can be, for example, in the form of tablets, caplets, or microtablets. The compact can be prepared by any means known in the art. For example, if the compact is in the form of microtablets, the microtablets can be made by compressing the composition described above using any known method, such as using a rotary tablet press equipped with a multi-tip tooling and having concave tips.

Multi-tip tableting tools, for example, can be used. For example, a multi-tip tool having from about 16 tips to about 40 tips using, for example, about 2 mm diameter tips. In this situation, applied compressing force can be expressed as an average kN/tip. For example, an applied compressing force of 2 kN used with a 16 multi-tip tool yields an applied compressing force of about 0.125 kN/tip. Similarly, an applied compressing force of about 15 kN used with a 16 multi-tip tool yields an applied compressing force of about 0.94 kN per tip.

The microtablets can have a mean diameter (excluding any coatings), for example, ranging from about 1 mm to about 3 mm. For example, the microtablets can have a mean diameter ranging from about 1 mm to about 2.5 mm. The microtablets can have a mean diameter of about 1.0 mm, about 2.0 mm, or about 3.0 mm.

Compact tensile strength can be determined by any means known in the art. For example, the following protocol could be employed. First, compact(s) are compressed to about 360 mg weight using an instrumented rotary tablet press equipped to measure compression force with round flat tooling of approximately 10 mm diameter. Next, measure the diametrial crushing strength using a suitable tablet hardness tester and then calculate tensile strength by the procedure reported by Newton (Newton, J. M., Journal of Pharmacy and Pharmacology, 26: 215-216 (1974)). See also Pandeya and Puri, KONA Powder and Particle Journal, 30: 211-220 (2013), Jarosz and Parrott, J. Pharm. Sci. 72(5):530-535 (1983), and Podczeck, Intl. J. Pharm. 436:214-232 (2012).

The composition, in the form of a compact, can have a tensile strength equal to or greater than 1.5 MPa at an applied or compaction pressure of about 100 MPa. For example, the tensile strength can range from about 2.0 to about 5.0 MPa (e.g., from about 2.5 to about 4.5 MPa, from about 3.0 to about 4.5 MPa or from about 3.5 to about 4.5 MPa) at an applied or compaction pressure of about 100 MPa. For example, the tensile strength can be about 4.0 MPa at an applied or compaction pressure of about 100 MPa.

The compact in the form of one or more microtablets produced using 16 multi-tip tooling can have a hardness or breaking strength or crushing strength ranging from about 8 N to about 35 N when the microtablet is formed by a compression force ranging from 2 kN to about 15 kN and the microtablet has a 2 mm diameter, a thickness of 2 mm, and a 1.8 mm radius of the convex surface. In one embodiment, microtablets each having a 2 mm diameter, a thickness of 2 mm, and a 1.8 mm radius of the convex surface have a hardness ranging from about 17 N to about 24 N for a compression force of about 4 kN to about 7 kN. The hardness can be, for example, of from about 23 N to about 27 N (e.g., about 24 N, about 25 N, or about 26 N) for a compression force of about 10 kN to about 15 kN. Hardness or breaking strength or crushing strength can be determined for example, using an Erweka tester or a Schleuniger tester as described in Lachman, L. et al., The Theory & Practice of Industrial Pharmacology (3rd ed. 1986), p. 298.

In some embodiments, the composition can be optionally coated or partially coated by one or more coatings. The coating(s) can be pH independent or pH dependent. The coating(s) can be, for example, enteric coatings, seal coatings, or combinations of enteric coatings and seal coatings.

The seal coating can contain, for example, one or more plasticizers, one or more copolymers, one or more polymers, or combinations thereof.

The plasticizer can be, for example, one or more of acetyltributyl citrate, acetyltriethyl citrate, benzyl benzoate, cellulose acetate phthalate, chlorbutanol, dextrin, dibutyl phthalate, dibutyl secacate, diethyl phthalate, dimethyl phthalate, glycerin, glycerin monostearate, hypromellose phthalate, mannitol, mineral oil an lanolin alcohols, palmitic acid, polyethylene glycol, polyvinyl acetate phthalate, propylene glycol, 2-pyrrolidone, sorbitol, stearic acid, triacetin, tributyl citrate, triethanolamine, and triethyl citrate.

The copolymer can be, for example, a methacrylic acid-methacrylate copolymer or a methacrylic acid-ethylacrylate copolymer.

Additionally, the seal coating can contain one or more polymers, for example, cellulose derivatives such as hydroxyethyl cellulose, hydroxypropyl cellulose, hydroxypropyl and methylcellulose, polyvinylpyrrolidone, a polyvinylpyrrolidone/vinyl acetate copolymer, ethyl cellulose, and ethyl cellulose aqueous dispersions (AQUACOAT®, SURELEASE®), EUDRAGIT® RL 30 D, OPADRY®, EUDRAGIT® S, EUDRAGIT® L, and the like.

If present in the seal coating, the total amount of one or more copolymer(s) and/or one or more polymer(s) in the seal coating can range, for example, from a positive amount greater than 0% w/w to about 100% w/w, based on the weight of the seal coating. The amount of one or more copolymer(s) and/or one or more polymer(s) in the seal coating can range, for example, from about 10% w/w to about 100% w/w, from about 20% w/w to about 100% w/w, from about 30% w/w to about 100% w/w, from about 40% w/w to about 100% w/w, from about 50% w/w to about 100% w/w, from about 60% w/w to about 100% w/w, from about 70% w/w to about 100% w/w, from about 80% w/w to about 100% w/w, or from about 90% w/w to about 100% w/w, based on the weight of the seal coating.

The amount of one or more copolymer(s) and/or one or more polymer(s) in the seal coating can be, for example, about 10% w/w, about 20% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, or about 95% w/w, based on the weight of the seal coating.

If present in the seal coating, the mean amount of plasticizer in the seal coating can range, for example, from a positive amount greater than 0% w/w to about 70% w/w, based on the weight of the seal coating.

The enteric coating can contain, for example, one or more plasticizers, one or more fillers, one or more lubricants, one or more copolymers, one or more polymers, and any combinations thereof.

The plasticizer(s) in the enteric coat can be the same or different than any plasticizer(s) in a seal coat, if present, and can be one of more of the plasticizers listed above.

The filler(s) in the enteric coat can be the same or different than any filler(s) in the composition. Additionally, the filler(s) in the enteric coat can be the same or different than any filler(s) in a seal coat, if present, and can be one or more of the fillers listed above.

The lubricant(s) in the enteric coat can be the same or different than any lubricant(s) in the composition. Additionally, the lubricant(s) in the enteric coat can be the same or different than the copolymer(s) in a seal coat, if present, and can be one or more of the lubricants listed above. In one embodiment, the lubricant is talcum that is optionally micronized.

The copolymer(s) in the enteric coat can be the same or different than the copolymer(s) in a seal coat, if present, and can be one or more of the copolymer(s) listed above. In one embodiment, the enteric coat contains one or more of a methyl acrylate-methyl methacrylate-methacrylic acid copolymer (EUDRAGIT® FS 30 D), a methacrylic acid-methyl methacrylate copolymer and a methacrylic acid-ethyl acetate copolymer.

The enteric polymers used in this invention can be modified by mixing or layering with other known coating products that are not pH sensitive. Examples of such coating products include ethyl cellulose, hydroxylpropyl cellulose, neutral methacrylic acid esters with a small portion of trimethylammonioethyl methacrylate chloride, sold currently under the trade names EUDRAGIT® RS and EUDRAGIT® RL; a neutral ester dispersion without any functional groups, sold under the trade names EUDRAGIT® NE 30 D; and other pH independent coating products.

The total amount of the copolymer(s) and/or polymer(s) in the enteric coating can range, for example, from about 25% w/w to about 100% w/w, based on the weight of the enteric coating.

If present in an enteric coating, the total amount of lubricant(s) in the enteric coating can range, for example, from a positive amount greater than 0% w/w to about 58% w/w, based on the weight of the enteric coating.

If present in an enteric coating, the total amount of filler(s) in the enteric coating can range, for example, from a positive amount greater than 0% w/w to about 5.0% w/w, based on the weight of the enteric coating.

Solvents for applying the coating materials, can be, but are not limited to, water, acetone, hexane, ethanol, methanol, propanol, isopropanol, butanol, isobutanol, sec-butanol, tert-butanol, dichlormethane, trichloromethane, chloroform, and the like.

Coatings can be applied by any known means, including spraying. In some embodiments, the compositions are coated or partially coated with one or more seal coatings, for example one, two, three or more seal coatings. In some embodiments, the compositions are coated or partially coated with one or more enteric coatings, for example one, two, three or more enteric coatings. In some embodiments, the compositions are coated with one or more seal coatings and one or more enteric coatings. In some embodiments, the compositions are coated with one seal coating and one enteric coating.

In one embodiment, the composition is in the form of a dosage form, such that one composition provides the total DMF dose. In other embodiments, the dosage form contains multiple compositions to provide the total DMF dose. For example, a dosage form may contain multiple compacts, such as microtablets, to provide the desired total DMF dose.

If the dosage form contains multiple compacts, such as multiple microtablets, to provide the required total DMF dose, the compacts in the dosage form can differ from one another. For example, the dosage form can contain two or more different microtablet types (e.g., the capsule can contain one group of microtablets coated with only an enteric coating and a second group of microtablets coated with only a seal, coating, or one group coated with an enteric coating with a lower pH release and the other coated with an enteric coating with a higher pH release).

In some embodiments, the composition is placed in a capsule. In other embodiments, the composition, in the form of microtablets, is placed in a capsule. The capsule can contain, for example, from about 30 microtablets to about 60 microtablets, from about 35 microtablets to about 55 microtablets, or from about 40 microtablets to about 50 microtablets (e.g., about 44, about 45, about 46, about 47, or about 48 microtablets).

The dosage form can be administered, for example, to a mammal, or a mammal in need thereof. The dosage form can be administered, for example, to a human or a human in need thereof.

The dosage form can be administered, for example, 1×, 2×, 3×, 4×, 5×, or 6× per day. One or more dosage form can be administered, for example, for one, two, three, four, five, six, or seven days. One or more dosage forms can be administered, for example, for one, two, three, or four weeks. One or more dosage forms can be administered, for example, for one, two, three, four, five, six, seven, eight, nine, ten, eleven, twelve months or longer. One or more dosage forms can be administered until the patient, subject, mammal, mammal in need thereof, human, or human in need thereof, does not require treatment, prophylaxis, or amelioration of any disease or condition such as, for example, neurodegenerative disorders. Neurodegenerative disorders include, for example, MS (which includes relapsing remitting multiple sclerosis (RRMS), secondary progressive multiple sclerosis (SPMS), primary progressive multiple sclerosis (PPMS), progressive relapsing multiple sclerosis (PRMS)), amyotrophic lateral sclerosis (ALS), Alzheimer's disease, Parkinson's disease, and any combination thereof.

In some embodiments, a method according to the invention comprises orally administering a dosage form that provides a total amount of about 60 mg to about 1000 mg of dimethyl fumarate. The dosage form can, for example, contain a total amount of DMF effective for treatment, prophylaxis, or amelioration of multiple sclerosis. The effective amount can range, but is not limited to, a total amount of about 60 mg to about 800 mg DMF, about 60 mg to about 720 mg DMF, 60 mg to about 500 mg DMF, about 60 mg to about 480 mg DMF, about 60 mg to about 420 mg DMF, about 60 mg to about 360 mg DMF, about 60 mg to about 240 mg DMF, about 60 mg to about 220 mg DMF, about 60 mg to about 200 mg DMF, about 60 mg to about 180 mg DMF, about 60 mg to about 160 mg DMF, about 60 mg to about 140 mg DMF, about 60 mg to about 120 mg DMF, about 60 mg to about 100 mg DMF, about 60 mg to about 80 mg DMF, about 80 mg to about 480 mg DMF, about 100 mg to about 480 mg DMF, about 120 mg to about 480 mg DMF, about 140 mg to about 480 mg DMF, about 160 mg to about 480 mg DMF, about 180 mg to about 480 mg DMF, about 200 mg to about 480 mg DMF, about 220 mg to about 480 mg DMF, about 240 mg to about 480 mg DMF, about 300 mg to about 480 mg DMF, about 360 mg to about 480 mg DMF, about 400 mg to about 480 mg DMF, about 450 mg to about 500 mg DMF, about 480 mg to about 500 mg DM F, about 80 to about 400 mg DMF, about 100 to about 300 mg DMF, about 120 to about 180 mg DMF, or about 140 mg to about 160 mg DMF.

The dosage form can contain, but is not limited to, a total amount of DMF of about 60 mg DMF, about 80 mg DMF, about 100 mg DMF, about 120 mg DMF, about 140 mg DMF, about 160 mg DMF, about 180 mg DMF, about 200 mg DMF, about 220 mg DMF, about 240 mg DMF, about 260 mg DMF, about 280 mg DMF, about 300 mg DMF, about 320 mg DMF, about 340 mg DMF, about 360 mg DMF, about 380 mg DMF, about 400 mg DMF, about 420 mg DM F, about 450 mg DMF, about 480 mg DMF, or about 500 mg DMF.

In some embodiments, DMF is the only active ingredient in the composition.

For the treatment of MS (e.g., relapsing forms of MS such as RRMS), the dosage form administered to the patients or patients in need thereof can be a capsule with microtablets containing DMF as the only active ingredient wherein the effective amount is about 480 mg DMF per day, and the patients can receive the effective amount, i.e., 240 mg DMF BID, in the form of two capsules a day, to be taken orally.

DMF is known to cause flushing and gastrointestinal (GI) side effects in certain patients. While the side effects generally subside soon after patients start on the treatment, the starting dose is 120 mg DMF BID orally for the first 7 days. The dose can be increased to the effective dose of 240 mg DMF BID (i.e., 480 mg DMF per day). For those patients who experience GI or flushing side effects, taking DMF with food can improve tolerability.

In a healthy volunteer study, administration of 325 mg non-enteric coated aspirin 30 minutes prior to DMF dosing is found to reduce the occurrence and severity of flushing in the participating subjects. Some patients who experience flushing with gastrointestinal side effects may reduce the dose to 120 mg DMF BID temporarily. Within a month, the effective dose of 240 mg DMF BID should be resumed.

In one embodiment, patients administered a dosage form described above may take one or more non-steroidal anti-inflammatory drugs (e.g., aspirin) before (for example, 10 minutes to an hour, e.g., 30 minutes before) taking the dosage form described above. In one embodiment, the patient administered the dosage form takes the one or more non-steroidal anti-inflammatory drugs (e.g., aspirin) to reduce flushing. In another embodiment, the one or more non-steroidal anti-inflammatory drugs is selected from a group consisting of aspirin, ibuprofen, naproxen, ketoprofen, celecoxib, and combinations thereof. The one or more non-steroidal anti-inflammatory drugs can be administered in an amount of about 50 mg to about 500 mg before taking the dosage form described above. In one embodiment, a patient takes 325 mg aspirin before taking each dosage form described above.

In some embodiments, patients orally administered one or more non-steroidal anti-inflammatory drugs (e.g., aspirin) before taking the dosage form described above exhibit the same pharmacokinetic properties (e.g., Cₘₐₓ and AUC) as patients orally administered the dosage form described above without administering one or more non-steroidal anti-inflammatory drugs (e.g., aspirin).

In one embodiment, patients with multiple sclerosis are administered a capsule containing 240 mg DMF, twice daily for a total daily dose of 480 mg, wherein the capsule contains multiple microtablets comprising about 43% w/w to about 95% w/w (e.g., from about 50% to about 80% w/w) DMF, by weight of the microtablets without any coatings. In one embodiment, the microtablets are first coated with a seal coat and then coated with an enteric coat. In one embodiment, the patients administered the capsular dosage form exhibit one or more of the pharmacokinetic parameters described above.

The following examples are illustrative and do not limit the scope of the claimed embodiments.

### EXAMPLES

### Example 1: Compositions Containing 42% and 65% w/w of Dimethyl Fumarate

Dimethyl fumarate (DMF), croscarmellose sodium, talc, and silica colloidal anhydrous were mixed together to form a blend according to the amounts as described in Table 1 below. The blend was then passed through a screen (e.g., screen with 800 micron aperture) and microcrystalline cellulose (PROSOLV SMCC® HD90) was added to the blend and mixed. Magnesium stearate was added to the blend and the blend was remixed. The resulting blend was then compressed on a suitable rotary tablet press equipped with 16 multi-tip tooling having 2 mm round concave tips.

Table 1 below provides the weight percentages of ingredients present in two type of microtablets made using the method described above. A size 0 capsule containing microtablets made with blend A contain about 120 mg of DMF whereas the same size capsule containing microtablets made with blend B contain about 240 mg of DMF.

TABLE 1

| | Composition, % w/w | |
|---|---|---|
| Ingredients | Blend A | Blend B |
| DMF | 42 | 65 |
| Croscarmellose | 5.0 | 5.0 |
| sodium | | |
| Prosolv SMCC ® | — | 29 |
| HD90 | | |
| Avicel PH200 | 44 | — |
| Magnesium Stearate | 1.7 | 0.5 |
| Talc | 6.6 | — |
| Silica colloidal | 0.86 | 0.60 |
| anhydrous | | |
| Total | 100 | 100 |

Because of the concave shape of the microtablets, tensile strength of microtablets made with blends A and B were evaluated by measuring the tensile strength of the corresponding 10 mm round cylindrically shaped compacts. The corresponding compacts were made by compressing about 360 mg of blends A and B using an instrumented rotary tablet press equipped to measure compression force with round flat tooling of approximately 10 mm diameter. Diametrial crushing strength of the compacts made from blends A and B was then measured using a suitable tablet hardness tester (e.g., Key International hardness tester HT500) and tensile strength was then calculated by the procedure reported by Newton (Newton, J. M., Journal of Pharmacy and Pharmacology, 26: 215-216 (1974)).

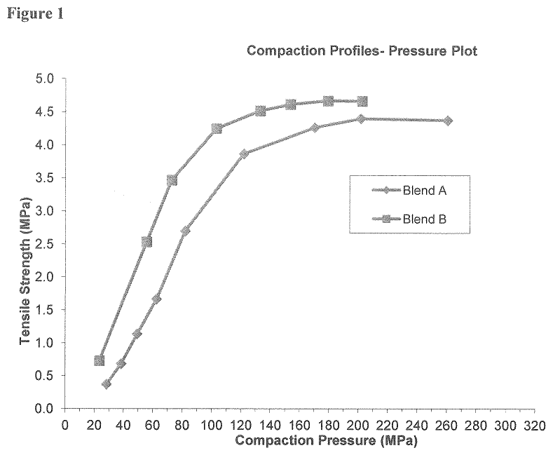

FIG. 1 shows the tensile strength of compacts made with blend A and blend B. Despite having less excipients such as microcrystalline cellulose (a binder), the tensile strength of compact made with blend B unexpectedly shows similar (or even some improvement) over that made with blend A. Tensile strength of microtablets made with blends A and B reflect the same trend.

### Example 2: Formation of Capsules Containing Microtablets

Dimethyl fumarate, croscarmellose sodium, talcum and colloidal silicon anhydrous are mixed together to form a blend according to the amounts described in Table 2 below. The blend is passed through a screen. A suitable grade of microcrystalline cellulose, for example, PROSOLV SMCC® 90 or PROSOLV SMCC® HD90 is added to the blend and mixed. Magnesium stearate is added to the blend and the blend is remixed.

The blend is then compressed on a suitable rotary tablet press equipped with multi-tip tooling (e.g., a 16 multi-tip tooling) having 2 mm round concave tips. The resulting 2 mm sized microtablets are coated with a solution of methacrylic acid-methyl methacrylate copolymer and triethyl citrate in isopropanol (see amounts in Table 2 below). The coated microtablets are then coated with a second layer of coating consisting of methacrylic acid-ethylacrylate copolymer, polysorbate 80, sodium lauryl sulfate, triethyl citrate, simethicone, and talcum micronized suspended in water (see amounts in Table 2 below).

The desired amount of coated microtablets are encapsulated in a two piece hard gelatin capsule using a capsule machine. For example, coated microtablets are encapsulated in a capsule such that the amount of dimethyl fumarate is about 240 mg per capsule.

In Table 2 below, % w/w is based on the total weight of the coated microtablet (e.g., in this table, % w/w includes the weight contributions of the coatings).

TABLE 2

| | Net capsule content, % w/w of the capsule components | | | | | | | | | |
|---|---|---|---|---|---|---|---|---|---|---|
| | Example No. | | | | | | | | | |
| Ingredients | 1 | 2 | 3 | 4 | 5 | 6 | 7 | 8 | 9 | 10 |
| Dimethyl fumarate | 43.01 | 72.30 | 58.40 | 54.08 | 83.60 | 73.90 | 39.50 | 65.00 | 33.90 | 42.00 |
| Croscarmellose | 1.26 | 0.33 | 3.72 | 4.17 | 0.46 | 0.89 | 4.43 | 4.00 | 4.24 | 3.00 |
| sodium | | | | | | | | | | |
| Microcrystalline | 41.82 | 15.91 | 17.31 | 23.57 | 7.00 | 9.42 | 31.31 | 13.66 | 37.18 | 35.79 |
| Cellulose | | | | | | | | | | |
| Magnesium Stearate | 1.05 | 0.25 | 0.69 | 0.41 | 0.26 | 0.63 | 1.32 | 0.40 | 1.41 | 0.48 |
| Silica colloidal | 1.21 | 0.22 | 0.78 | 0.97 | 0.43 | 0.29 | 0.69 | 0.40 | 0.73 | 0.68 |
| anhydrous | | | | | | | | | | |
| Methacrylic acid | 1.01 | 1.27 | 0.98 | 1.51 | 0.11 | 1.66 | 1.87 | 1.21 | 1.55 | 1.32 |
| methyl acrylate | | | | | | | | | | |
| copolymer | | | | | | | | | | |
| Methacrylic acid | 6.23 | 4.98 | 11.12 | 8.97 | 4.34 | 8.21 | 9.93 | 7.72 | 9.04 | 9.98 |
| ethyl acrylate | | | | | | | | | | |
| copolymer | | | | | | | | | | |
| Triethyl citrate | 1.61 | 1.74 | 2.33 | 2.12 | 0.97 | 1.67 | 2.31 | 2.09 | 2.15 | 2.32 |
| Talc | 2.56 | 2.81 | 4.32 | 3.90 | 2.65 | 3.06 | 8.32 | 5.30 | 9.46 | 4.12 |
| Simethicone | 0.03 | 0.02 | 0.03 | 0.05 | 0.02 | 0.03 | 0.02 | 0.02 | 0.06 | 0.02 |
| polysorbate 80 | 0.15 | 0.11 | 0.24 | 0.20 | 0.11 | 0.18 | 0.22 | 0.14 | 0.21 | 0.21 |
| Sodium Lauryl | 0.06 | 0.06 | 0.08 | 0.07 | 0.05 | 0.06 | 0.08 | 0.06 | 0.06 | 0.08 |
| sulfate | | | | | | | | | | |

### Example 3: Formation of Microtablets

Dimethyl fumarate, croscarmellose sodium, talcum and colloidal silicon anhydrous were mixed together to form blends 1, 2, 4, 5, and 6 according to the amounts described in Table 3 below. Each blend was passed through a screen. Microcrystalline cellulose (PROSOLV SMCC® HD90) was added to the blends according to the amounts in Table 3 and mixed. Magnesium stearate was then added to each blend and the blend was remixed. Each blend was then compressed on a suitable rotary tablet press equipped with 16 multi-tip tooling having 2 mm round concave tips.

Blends 3, 7, 8, and 9 can be made using the same method as described above.

TABLE 3

| | Percent w/w Composition of the Core Microtablet | | | | | | | | |
|---|---|---|---|---|---|---|---|---|---|
| Ingredient | Blend 1 | Blend 2 | Blend 3 | Blend 4 | Blend 5 | Blend 6 | Blend 7 | Blend 8 | Blend 9 |
| Dimethyl fumarate | 42.0 | 42.0 | 50.0 | 60.0 | 65.0 | 70.0 | 75.0 | 85.0 | 95.0 |
| Croscarmellose | 5.0 | 5.0 | 3.0 | 5.0 | 5.0 | 5.0 | 1.0 | 1.0 | 0.4 |
| sodium | | | | | | | | | |
| Microcrystalline | 44.0 | 50.0 | 43.0 | 32.0 | 28.3 | 23.0 | 22.0 | 13.0 | 4.0 |
| Cellulose | | | | | | | | | |
| Magnesium Stearate | 1.7 | 1.7 | 0.5 | 1.7 | 0.5 | 1.3 | 0.4 | 0.4 | 0.4 |
| Silica colloidal | 0.9 | 1.2 | 1.5 | 1.0 | 1.2 | 0.9 | 0.6 | 0.5 | 0.5 |
| anhydrous | | | | | | | | | |
| Talc | 6.6 | — | 2.0 | — | — | — | 1.0 | — | — |
| total | 100 | 100 | 100 | 100 | 100 | 100 | 100 | 100 | 100 |

### Example 4: Compacts Containing 42% w/w, 60% w/w, and 70% w/w Dimethyl Fumarate and Control Compacts

Dimethyl fumarate, croscarmellose sodium, and silica colloidal anhydrous were blended together to form a blend. The blend was passed through a screen. A suitable grade of microcrystalline cellulose was added to the screened blend and blend was mixed. A suitable grade of microcrystalline cellulose, is, for example PROSOLV SMCC® 90, having an average particle size by laser diffraction of about 60 μm and a bulk density ranging from about 0.38 to about 0.50 g/cm³. Magnesium stearate was added to the mixed blend and remixing was effected.

The respective blended materials were compressed on a suitable rotary press (e.g., a rotary tablet press) to form compacts (10 mm cylindrical compacts).

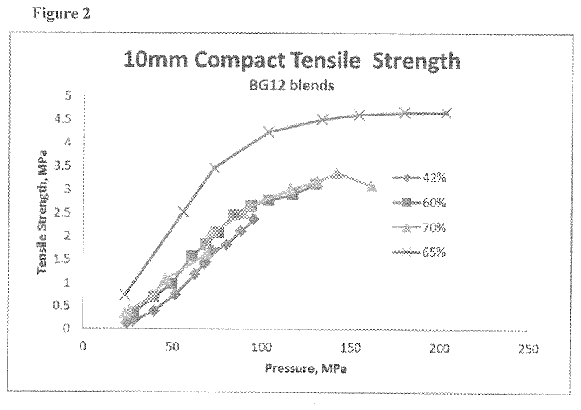

The following table provides percentages for representative compacts made by this process. Tensile strength of the DMF-containing compacts (i.e., compacts containing 42%, 60%, and 70% w/w of DMF) were measured according to the method as described in Example 1 above and shown in FIG. 2. Tensile strength of blend B in Example 1 (containing 65% w/w of DMF) was also shown in FIG. 2.

TABLE 4

| Ingredients | 42% | 60% | 70% |
|---|---|---|---|
| Dimethyl fumarate | 42 | 60 | 70 |
| Croscarmellose sodium | 5.0 | 5.0 | 5.0 |
| Microcrystalline Cellulose | 50 | 32 | 23 |
| Magnesium Stearate | 1.7 | 1.7 | 1.7 |
| Silica colloidal anhydrous | 1.2 | 1.0 | 0.9 |

### Example 5: Compositions Containing 65% w/w, 95% w/w, and 99.5% w/w Dimethyl Fumarate

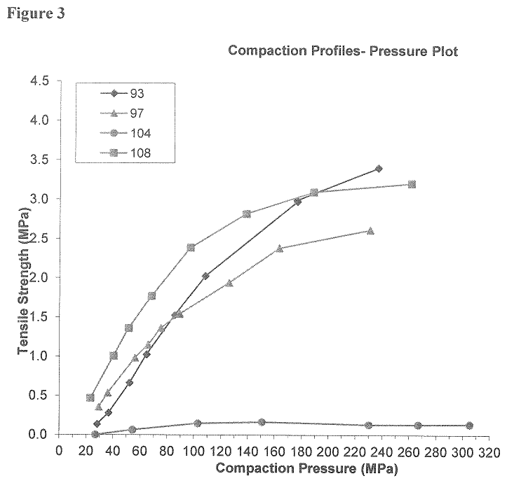

Four DMF-containing blends were prepared according to the method as described in Example 4 above with the amounts as described in Table 5 below. Tensile strength of the blends was also measured as described above and shown in FIG. 3. Flowability was measured as described in Example 6 below.

TABLE 5

| | Composition, % by weight | | | |
|---|---|---|---|---|
| Ingredients | Blend 93 | Blend 97 | Blend 104 | Blend 108 |
| Dimethyl fumarate | 65 | 95 | 99.5 | 95 |
| Prosolv SMCC 90 | 28.9 | 2 | — | 2 |
| Croscarmellose | 5 | 2 | — | 2 |
| Sodium | | | | |
| Silica colloidal, | 0.6 | 0.6 | — | 0.6 |
| anhydrous | | | | |
| Magnesium | 0.5 | 0.4 | 0.5 | 0.4 |
| stearate | | | | |
| Particle size of | 14% < | 14% < | 15% < 250μ | 84% < 250μ |
| dimethyl fumarate | 250μ | 250μ | | |
| Flodex(mm) | 4 | 4 | 4 | 6 |
| Bulk density(g/ml) | 0.66 | 0.66 | 0.74 | 0.69 |
| Tapped | 0.79 | 0.78 | 0.83 | 0.82 |
| density(g/ml) | | | | |
| Compressibility, % | 17 | 16 | 17 | 17 |

### Example 6: Measuring Flowability of Powder Blends

A powder sample (e.g., 50 g) was loaded into the cylinder on a FLODEX device such that the powder was within about 1 cm from the top of the cylinder. A minimum of 30 seconds was allowed to pass before testing commences. Starting with a 16 mm flow disk, the release lever was slowly turned until the closure dropped open without vibration. The test was positive when the open hole at the bottom was visible when looking down from the top. If a positive result was obtained, the test was repeated with smaller and smaller disk holes until the test was negative. For negative results, the size of the flow disk hole was increased until the test was positive. The flowability index is the diameter of the smallest hole through which the sample will pass for three successive tests. Results are shown below.

The compressibility index was arrived at, for example, as follows: Powder was placed in a container and the powder's unsettled apparent volume (Vₒ) was noted. Next, the powder was tapped until no further volume changes occur. At this point, the final tapped volume of the powder was measured (V_{f}). The compressibility index was calculated using the following formula: ((Vₒ−V_{f})/Vₒ)×100%. Compressibility indexes (e.g., Carr Indexes) are provided in the table below:

TABLE 6

| | Amount of | | | | Flodex |
|---|---|---|---|---|---|
| | DMF (% w/w) | Bulk | Tapped | Carr Index | Minimum |
| | in the | Density | Density | (Compressibility, | Opening |
| | Composition | g/ml | g/ml | %) | (mm) |
| 1 | 42 | 0.44 | 0.54 | 18 | — |
| 2 | 42 | — | — | — | 18 |
| 3 | 60 | 0.54 | 0.67 | 19 | 12 |
| 4 | 65 | 0.52 | 0.66 | 20 | 14 |

### Example 7: Measuring PK Parameters and Evaluating Bioequivalence of Pharmaceutical Compositions Containing 120 mg DMF and 240 mg DMF in Capsules Containing Microtablets

Eighty-one subjects were enrolled and randomized to a treatment sequence.

Sequence 1 having 41 subjects in which the reference product was given orally as 2 capsules each containing 120 mg DMF (42% w/w) (Dosing Period 1), followed by the test product of DMF 240 mg (65% w/w), given orally as a single capsule (Dosing Period 2); or

Sequence 2 having 40 subjects in which the test product of DMF 240 mg was given orally as a single capsule (Dosing Period 1), followed by the reference product, given orally as 2 capsules, each containing 120 mg DMF (Dosing Period 2).

All subjects in both treatment sequences completed Dosing Period 2, and 77 subjects completed Dosing Period 2. Seventy-seven subjects completed the study. All subjects (41) in Sequence 1 completed the study. Thirty-six subjects in Sequence 2 completed the study.

Four subjects in Sequence 2 withdrew from the study during the washout interval prior to Dosing Period 2: 2 withdrew due to adverse effects, 1 withdrew consent due to family reasons, and 1 withdrew due to Investigator decision.

The study population consisted of young adults, balanced between male (57%) and female (43%) subjects. Most of the subjects were white (85%). Across all subjects, the median age was 28 years with a range from 19 to 56 years. The median weight was 73.6 kg, ranging from 48.8 to 96.5 kg.

The PK population, defined as all subjects who received at least one of the two treatments and with at least one measurable MMF concentration, included 77 subjects dosed with the reference product and 81 subjects dosed with the test product.

PK samples were drawn during Dosing Periods 1 and 2 for each treatment sequence per the following schedule: −15 min., 30 min., 60 min., 90 min., 2 hr., 3 hr., 4 hr., 5 hr., 6 hr., 7 hr., 8 hr., 10 hr., and 12 hr.

The plasma concentration-time profiles were analyzed via Non-Compartmental analysis (NCA) using WinNonLn, version 5.2.

AUC_{0→infinity} and Cₘₐₓ were the primary endpoints used to establish bioequivalence (BE). The two one-sided hypotheses at the α=0.05 level was to be tested by constructing the 90% confidence interval for the geometric mean ration of the test product (a single capsule of DMF 240 mg) to the reference product (2 capsules of 120 mg DMF). The standard 80% to 125% equivalence criterion was used.

After oral administration with test and reference products, the MMF concentration (monomethyl fumarate concentration)-time profiles displayed a short lagtime with a mean value less than 0.5 h. The concentration maximums (Cₘₐₓ) were attained at times (Tₘₐₓ) with means of approximately 2.5 hours for both reference and test product. The Cₘₐₓ values were very similar (means 2.34 mg/L for the reference product vs. 2.42 mg/L for the test product. The calculated AUC₀₋₁₂ values were also very similar (means 3.85 h·mg/L for the reference product vs. 3.93 h·mg/L for the test product), as were the extrapolated AUC_{0→infinity} values (means 3.87 h·mg/1 for the reference product vs. 3.98 h·mg/l for the test product).

This example showed that a single capsule of 240 mg DMF is bioequivalent to an equivalent dose administered as two capsules (120 mg DMF each).

### Example 8: Combination of DMF and Aspirin

A randomized, double-blind, placebo-controlled study in healthy adult volunteers was conducted in which a total of 56 subjects were randomized to receive 4 days' treatment with DMF 240 mg BID, DMF 240 mg TID, DMF 360 mg BID or placebo, with aspirin 325 mg or matching aspirin placebo administered 30 minutes before each DMF or DMF placebo dose. An additional 8 patients were assigned to a modified dosing group receiving DMF 120 mg or placebo 6 times daily (3 doses at hourly intervals in the morning and a further 3 doses at hourly intervals in the evening). There were 6 subjects per group, except for the modified dosing regimen, where an additional 2 subjects were assigned to placebo.

The pharmacokinetic profile of DMF was assessed by measuring the primary metabolite, MMF, in the plasma of subjects at 14 time points (Hours 0, 0.5, 1, 1.5, 2, 2.5, 3, 4, 5, 6, 7, 8, 9, 10) on day 1 and day 4. The concentration of MMF was determined by high pressure liquid chromatography with tandem mass spectrometry, using fumaric acid monomethyl ester as the internal standard. Further pharmacokinetic parameters were derived by non-compartmental analysis

Flushing severity was assessed by 2 validated subject-reported measures, the global Flushing Severity Scale (GFSS) and the Flushing Severity Scale (FSS), which are adapted from the flushing scale described in Norquist J M, et al. Curr Med Res Opin 23:1547-1560 (2007). Both of these measures rate flushing severity on a scale of 0-10, where 0=no flushing, 1-3=mild flushing, 4-6=moderate flushing, 7-9=severe flushing, and 10=extreme flushing. The GFSS is a visual-analogue scale measuring redness, warmth, tingling and itching of the skin experienced over the preceding 24 h. Subjects completed the GFSS immediately before the first dose of study drug (0 h) on days 1 to 4, again at 0 h on day 5 and once more at follow-up on day 11. On the FSS, subjects rated their overall flushing and 4 items describing specific flushing symptoms (redness, warmth, tingling, itching) at the time of administration of the questionnaire. The FSS scale was administered at 16 time points over 12 h (Hours 0, 0.5, 1, 1.5, 2, 2.5, 3, 4, 5, 6, 7, 8, 9, 10, 11, 12) on days 1 to 4 and once on day 5 (24 h after the first day 4 dose) to assess the quality and intensity of subject-reported flushing symptoms in real time. Subjects rated 5 items relating only to the period since they last answered the questionnaire and/or received study drug.

The severity of GI symptoms was assessed by means of 2 subject-reported instruments; the Overall GI Symptom Scale (OGISS) and the Acute GI Symptom Scale (AGIS). The OGISS and the AGIS use a similar 10-point scoring scale, where 0=no GI symptoms, 1-3=mild symptoms, 4-6=moderate symptoms, 7-9=severe symptoms and 10=extreme symptoms. The OGISS is a visual analogue scale that rates overall GI symptoms (diarrhea, vomiting, nausea, bloating/gas and stomach pain) experienced over the preceding 24 h. Subjects completed the OGISS as per the GFSS immediately before receiving study drug on days 1 to 4, again at 0 h on day 5 and once more at follow-up on day 11. The AGIS is a 5-item questionnaire measuring subjects' opinions of overall digestive symptoms, nausea, stomach pain, bloating/gas and vomiting since they last answered the questionnaire and/or received study drug. It was administered as per the FSS at 16 time points over 12 h on days 1 to 4 and once on day 5.

Laser Doppler perfusion was used as an exploratory quantitative measure of facial skin perfusion during flushing. This technique uses non-invasive imaging of superficial tissue blood perfusion, recorded as Blood Perfusion Units on a relative units scale. Laser Doppler perfusion was measured at the same 16 timepoints as the FSS.

The potential importance of PGD₂ in the flushing response was assessed by measuring metabolites of PGD₂ in plasma and urine. PGF_{2α}, 9α was measured in plasma samples drawn immediately before dosing and at 0.5, 1, 2, 3, 4, 6, 8, 10 and 12 h on days 1 and 4. The concentrations of PGF2_{α}, 9α were determined by gas chromatography-mass spectrometry (GC-MS) using d4-8-iso-PGF₂a as internal standard. The major urinary metabolite of PGD₂ is prostaglandin D-M (PGD-M). The levels of PGD-M in urine were assayed by GC-MS of pooled urine samples collected for 8 h on day −1, and between 0 h and 8 h on days 1 and 4. ¹⁸O-labeled PGD-M was used as internal standard.

The potential role of histamine in the flushing response was also evaluated; plasma histamine concentrations were determined by liquid chromatography-mass spectrometry from samples collected on days 1 and 4, using d4-histamine as the internal standard.

### Results

The MMF plasma concentration-time relationship (on day 1 and day 4) was irregular and subject to high inter-individual variability for all treatment groups. Pretreatment with aspirin had no apparent effect on the concentration-time profiles of any group. Although characterized by high inter-individual variation, median parameters were similar on day 1 and day 4 within each treatment group. Values for Tₘₐₓ were consistently higher with TID dosing, compared with BID dosing, as would be expected with carryover of the exposure from the first dose at the time of the second dose, which was administered 4 h later. Values for the AUC from 0-10 h (AUC₀₋₁₀ₕ) were dose-proportional and t_{1/2} values were very short (although the irregular shape of the concentration-time profiles made this parameter particularly difficult to interpret).

The pre-dose plasma MMF concentrations measured on day 4 were below the lower limit of quantification (LLOQ) except for 1 or 2 individuals per treatment group, who yielded very low values. The pre-dose carryover of exposure from previous doses did not exceed 2% of the subsequent maximum, i.e. there was no accumulation of exposure with any regimen. This was confirmed by comparison of the Cₘₐₓ and AUC₀₋₁₀ₕ values on day 1 and day 4 for each dosing arm with and without aspirin. There was no systematic increase in either of these parameters for 4 days. Nor were there any systematic changes in time-dependent parameters, such as T_{1/2}, Tₘₐₓ and lag time, over the 4 days, indicating that the shape and extent of the exposure did not change with any dosing regimen.

TABLE
Summary of median pharmacokinetic parameters

| | BG-12 240 mg BID | | BG-42 240 mg TID | |
|---|---|---|---|---|
| | Without | With | Without | With |
| | aspirin | aspirin | aspirin | aspirin |
| n | 6 | 6 | 6 | 6 |
| AUC₀₋₁₀ | | | | |
| (h · ng/mL) | | | | |
| Day 1 | 2800.0 | 3020.0 | 5075.0 | 5875.0 |
| Day 4 | 2865.0 | 2590.0 | 5815.0 | 5885.0 |
| Cₘₐₓ (ng/mL) | | | | |
| Day 1 | 1335.0 | 1625.0 | 1935.0 | 1970.0 |
| Day 4 | 1730.0 | 1135.0 | 2050.0 | 1995.0 |
| tₘₐₓ (hours) | | | | |
| Day 1 | 4.0 | 2.8 | 6.0 | 5.0 |
| Day 4 | 3.0 | 3.5 | 5.5 | 3.5 |
| t_{1/2} (hours) | | | | |
| Day 1 | 0.81 | 0.59 | 0.85 | 0.81 |
| Day 4 | 0.63 | 0.56 | 1.05 | 0.88 |
| Lag time | | | | |
| (hours) | | | | |
| Day 1 | 0.5 | 0.25 | 0.5 | 1.75 |
| Day 4 | 0.25 | 0.25 | 1.0 | 1.0 |

Mean GFSS scores, which measured the severity of flushing in the past 24 hours, were generally lower in subjects treated with DMF plus 325 mg aspirin than in subjects treated with DMF alone. Regardless of aspirin treatment assignment, GFSS scores were low (suggesting mild symptoms), decreased over time in a similar manner, and had returned to baseline by the time of follow-up on day 11 (7 days after the last dose of DMF). Flushing severity was rated highest on day 2 (first day of dosing), when mean GFSS scores in the DMF alone groups ranged from 1.5 to 3.5 (mild). Pretreatment with aspirin reduced the incidence and intensity of flushing in subjects who received DMF, with ratings on the day of greatest severity (day 2) ranging from 0.3 to 1.0. Scores for placebo groups (with or without aspirin) remained very low throughout the treatment period.

Similar to the findings with the GFSS, mean FSS scores, which measured real-time severity of flushing, were generally lower in subjects treated with DMF plus 325 mg aspirin, than in subjects treated with DMF alone. Since the FSS measures flushing severity at the time of administration of the instrument, the severity of flushing was generally rated highest on day 1 in all groups. Again, pretreatment with aspirin 325 mg appeared to decrease the intensity of flushing events in subjects treated with DMF. Overall, subjects treated with DMF alone rated flushing severity on the FSS as mild to moderate on day 1, with decreasing severity over time. Subjects in the DMF plus aspirin groups rated flushing severity as mild even on day 1, with decreasing severity over time. As with the GFSS, mean overall FSS scores for placebo groups (with or without aspirin) remained very low throughout the study.

Doppler perfusion profiles showed a high degree of inter-individual variability in median percentage changes from baseline; however, the magnitude of the response was decreased by aspirin pretreatment. Visual inspection of the mean Doppler perfusion profiles for subjects treated with DMF alone showed that the peaks appeared to correspond to the times associated with maximum plasma MMF exposure.

Mean OGISS scores, which measured GI symptoms over the past 24 h, were low (≤1.0) throughout the study for all treatment groups and were reflective of mild symptoms. There were no apparent treatment- or dose-related differences in GI symptoms, and aspirin did not appear to modify the incidence or intensity of symptoms on this scale.

As with the OGISS, mean AGIS scores, which measured the overall GI symptoms since last assessment or study drug administration, were low (≤0.2) for all treatment groups and were reflective of mild symptoms. There were no apparent treatment- or dose-related differences in GI symptoms and pretreatment with aspirin did not appear to modify the reporting of acute GI symptoms on this scale.

Plasma concentrations of 9α, 11β-PGF_{2α} (the major metabolite of PGD_{2α}) were elevated at around 2-4 h on day 1 in subjects treated with DMF alone. On day 4, no major elevations of this metabolite in plasma were evident. Subjects treated with DMF plus aspirin, showed no elevation in their plasma concentrations of 9α, 11β-PGF_{2α} on either of the assessment days.

There was an elevation in urine PGD-M (the major urine metabolite of PGD_{2α}) levels from baseline to day 1 in some subjects treated with DMF alone, which returned to near baseline by day 4 for all subjects. This elevation was not seen in the placebo groups, or in subjects treated with DMF plus aspirin.

### Example 9: Synthesis of (E)-O,O′-(dimethylsilanediyl)dimethyl difumarate (Compound 11)

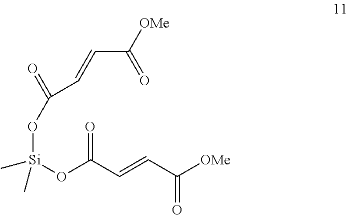 [SMILES: COC(=O)/C=C/C(=O)O[Si](C)(C)OC(=O)/C=C/C(=O)OC]

### Step 1: Preparation of Dimethylsilanediyl Diacetate 11B

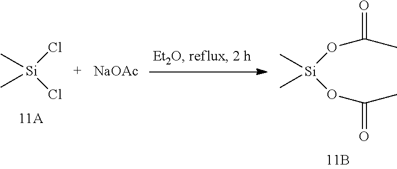

To a slurry of sodium acetate (8.2 g, 100 mmol, 2.0 equiv.) in anhydrous diethyl ether (40 mL) was slowly added a solution of dimethyldichloro silane 11A (6.45 g, 50 mmol, 1.0 equiv.) in anhydrous diethyl ether (10 mL). After addition was completed, the mixture was heated at reflux for 2 hours, and then filtered under N₂. The filtrate was concentrated under vacuum at 40° C. to give diacetate 11B as a colorless oil (6.1 g, 70%). ¹H NMR (400 MHz, CDCl₃) δ ppm: 2.08 (s, 6H), 0.48 (s, 6H).

### Step 2: Preparation of (E)-O,O′-(dimethylsilanediyl)dimethyl difumarate 11

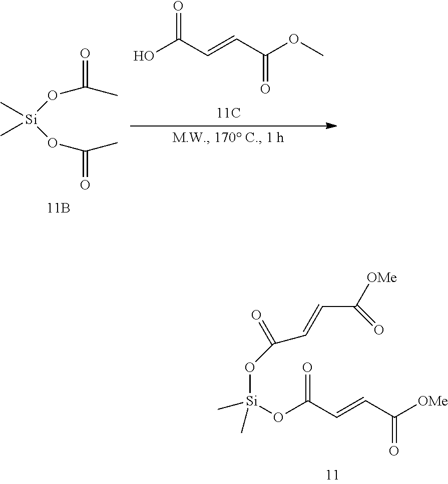

A mixture of 11B (2.0 mL, 12 mmol, 1.5 equiv.) and 11C (1.04 g, 8.0 mmol, 1.0 equiv.) in a sealed tube was heated at 170° C. with stirring under microwave condition for 1 hour. After cooling to 50° C., the mixture was transferred to a round bottom flask and the excess silica reactant 11B was removed under vacuum at 100° C. to afford compound 11 as brown oil (1.47 g, 60%). ¹H NMR (400 MHz, CDCl₃) δ ppm: 6.82-6.80 (m, 4H), 3.79 (s, 6H), 0.57 (s, 6H).

### Example 10: Synthesis of methyl ((trimethoxysilyl)methyl) fumarate (Compound 12)

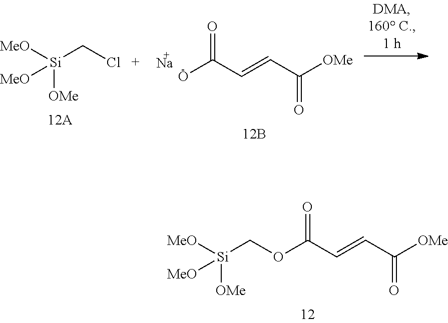

To a stirred solution of monomethyl fumarate (3.5 g, 27 mmol, 1.0 equiv.) in anhydrous THF (35 ml.) at room temperature was added sodium hydride (1.08 g, 27 mmol, 1.0 equiv.) in small portions. After addition, the mixture was heated to reflux for 3 hours, and then cooled to room temperature. The solid was collected by filtration and washed twice with diethyl ether, and further dried in vacuo to give 3.8 g of 12B (93%).

To a suspension of 12B (760 mg, 5.0 mmol, 1.0 equiv.) in dry DMA (5 mL) at 100° C. under nitrogen was added a solution of 12A (1.03 g, 6.0 mmol, 1.2 equiv.) in dry DMA (1 mL) dropwise. The resulting mixture was heated to 160° C. and stirred for 1 hour, and then cooled to room temperature. The solid was filtered, and the filtrate was evaporated under reduced pressure to give the titled compound 12, 513 mg (37%), as a red viscous liquid.

¹H NMR (400 MHz, CDCl₃) δ ppm: 6.90-6.86 (m, 2H), 3.97 (s, 2H), 3.82 (s, 3H), 3.62 (s, 9H).

### Example 11: Synthesis of methyl ((trihydroxysilyl)methylfumarate (Compound 13)

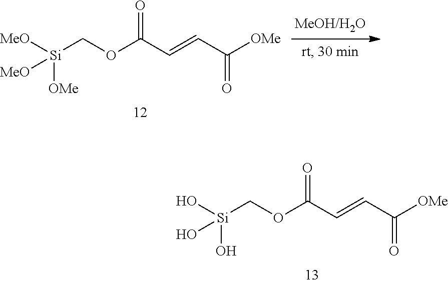 [SMILES: COC(=O)/C=C/C(=O)OC[Si](OC)(OC)OC ; COC(=O)/C=C/C(=O)OC[Si](O)(O)O]

To a solution of 12 (1.0 g, 3.8 mmol, 1.0 equiv., prepared in Example 2) in MeOH (10 mL) at room temperature was added water (341 mg, 19.0 mmol, 5.0 equiv.) dropwise. After addition, the mixture was stirred at room temperature for 30 minutes, with white solids precipitated out. The solids were collected through filtration, washed with methanol three times, and dried at 60° C. in vacuo, to provide the titled compound 13, 500 mg (59%), as a white solid.

¹H NMR (400 MHz, DMSO-d6) δ ppm: 6.79-6.74 (m, 2H), 3.91-3.58 (m, 6H), 3.18-3.15 (m, 2H).

### Example 12: Synthesis of trimethyl (methylsilanetriyl) trifumarate (Compound 14)

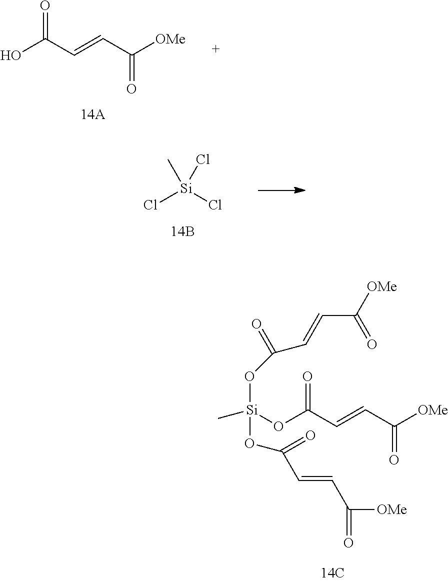

Following the procedure described in Scheme 9, monomethyl fumarate 14A would react with trichloromethane-silane 14B in refluxing toluene or hexanes with a catalytic amount of triethylamine to provide (2′E, 2″E)-trimethyl O,O′,O″-(methylsilanetriyl) trifumarate 14C.

All publications, patents, and patent applications referenced herein are incorporated by reference in their entireties.

In the event of a conflict between terms herein and terms in incorporated references, the terms herein control.

What is claimed is:

1. A composition comprising dimethyl fumarate and one or more excipients, wherein the total amount of dimethyl fumarate in the composition ranges from about 43% w/w to about 95% w/w.

2. The composition of claim 1, wherein the total amount of dimethyl fumarate in the composition ranges from about 50% w/w to about 80% w/w.

3. The composition of claim 2, wherein the total amount of dimethyl fumarate in the composition is about 65% w/w.

4. The composition of claim 1, wherein the total amount of dimethyl fumarate in the composition is about 95% w/w.

5. The composition of any one of claims 1-4, wherein the one or more excipients is selected from the group consisting of one or more fillers, one or more disintegrants, one or more glidants, one or more lubricants, and combinations thereof.

6. The composition of claim 4, wherein the one or more excipients is selected from the group consisting of microcrystalline cellulose, croscarmellose sodium, colloidal silica anhydrous, magnesium stearate, talc, and combinations thereof.

7. The composition of any one of claims 1-6, wherein the composition is in the form of a compact.

8. The composition of claim 7, wherein the compact has a tensile strength that is equal to or greater than about 1.5 MPa at an applied pressure of about 100 MPa.

9. The composition of claim 7, wherein the compact has a tensile strength that is equal to or greater than about 3.0 MPa at an applied pressure of about 100 MPa.

10. The composition of claim 7, wherein the compact is in the form of a microtablet.

11. The composition of claim 10, wherein dimethyl fumarate is the only active ingredient in the composition.

12. The composition of any one of claim 10, wherein an uncoated microtablet has a mean diameter ranging from about 1 mm to about 3 mm.

13. The composition of claim 10, wherein the microtablet is coated with one or more of methacrylic acid-methyl acrylate copolymer, methacrylic acid-ethyl acrylate copolymer, methacrylic acid-methylacrylate copolymer, ethyl cellulose, hydroxyl propyl cellulose, and methyl acrylate-methyl methacrylate-methacrylic acid copolymer.

14. A composition comprising about 43% w/w to about 95% w/w dimethyl fumarate, a total amount of about 3.5% w/w to about 55% w/w of one or more fillers, a total amount of about 0.2% w/w to about 20% w/w of one or more disintegrants, a total amount of about 0.1% w/w to about 9.0% w/w of one or more glidants, and a total amount of about 0.1% w/w to about 3.0% w/w of one or more lubricants.

15. The composition of claim 14, wherein the composition is in the form of a microtablet, said microtablet being uncoated and containing about 50% w/w to about 95% w/w dimethyl fumarate.

16. The composition of claim 15, wherein the composition contains about 65% w/w dimethyl fumarate.

17. A method of making a powder composition comprising combining about 43% w/w to about 95% w/w dimethyl fumarate, a total amount of about 3.5% w/w to about 55% w/w of one or more fillers, a total amount of about 0.2% w/w to about 20% w/w of one or more disintegrants, a total amount of about 0.1% w/w to about 9.0% w/w of one or more glidants, and a total amount of about 0.1% w/w to about 3.0% w/w of one or more lubricants to form the composition.

18. A composition comprising dimethyl fumarate and one or more excipients, wherein about 80% or higher of the dimethyl fumarate has a particle size of 250 microns or less.

19. The composition of claim 18, wherein about 97% or higher of the dimethyl fumarate has a particle size of 250 microns or less.

20. The composition of claim 1, wherein patients administered the composition exhibit a mean plasma monomethyl fumarate Tₘₐₓ of from about 1.5 hours to about 3.5 hours.

21. The composition of claim 1, wherein the composition is provided in a dosage form containing a total amount of about 240 mg dimethyl fumarate, wherein patients administered the dosage form twice daily, exhibit one or more pharmacokinetic parameters selected from the group consisting of (a) a mean plasma monomethyl fumarate Cₘₐₓ ranging from about 1.03 mg/L to about 2.41 mg/L and (b) a mean plasma monomethyl fumarate AUCₒᵥₑᵣₐₗₗ ranging from about 4.81 h·mg/L to about 11.2 h·mg/L.

22. The composition of claim 1, wherein the composition is provided in a dosage form containing a total amount of about 240 mg dimethyl fumarate, wherein patients administered the dosage form exhibit one or more pharmacokinetic parameters selected from the group consisting of (a) a mean plasma monomethyl fumarate Cₘₐₓ ranging from about 1.5 mg/L to about 3.4 mg/L, (b) a mean plasma monomethyl fumarate AUC₀₋₁₂ ranging from about 2.4 h·mg/L to about 5.5 h·mg/L, and (c) a mean AUC_{0-infinity} ranging from about 2.4 h·mg/L to about 5.6 h·mg/L.

23. A capsule comprising microtablets comprising dimethyl fumarate, wherein the total amount of dimethyl fumarate in an uncoated microtablet ranges from about 43% w/w to about 95% w/w.

24. The capsule of claim 23, wherein the microtablets are partially or fully enteric coated with at least one coating.

25. The capsule of claim 23, wherein the amount of dimethyl fumarate in the microtablets is about 60% w/w to about 70% w/w and the capsule contains about 35 to about 55 microtablets.

26. The capsule of claim 23, wherein the capsule contains a total amount of about 240 mg dimethyl fumarate, wherein patients administered the capsule exhibit one or more pharmacokinetic parameters selected from the group consisting of (a) a mean plasma monomethyl fumarate Tₘₐₓ of from about 1.5 hours to about 3.5 hours; (b) (a) a mean plasma monomethyl fumarate Cₘₐₓ ranging from about 1.5 mg/L to about 3.4 mg/L, (b) a mean plasma monomethyl fumarate AUC₀₋₁₂ ranging from about 2.4 h·mg/L to about 5.5 h·mg/L, and a mean AUC_{0-infinity} ranging from about 2.4 h·mg/L to about 5.6 h·mg/L.

27. A method for treating, prophylaxis, or amelioration of multiple sclerosis (MS) comprising orally administering to a subject in need thereof a therapeutically effective amount of dimethyl fumarate (DMF) and an amount of one or more non-steroidal anti-inflammatory drugs effective to reduce flushing.

28. The method of claim 27, wherein the one of more non-steroidal anti-inflammatory drug is aspirin.

29. A method of treating, prophylaxis, or amelioration of multiple sclerosis, comprising administering to a subject in need thereof a composition containing a compound, or a pharmaceutically acceptable salt thereof, that metabolizes to monomethyl fumarate wherein said administering the composition provides one or more of the following pharmacokinetic parameters: (a) a mean plasma monomethyl fumarate Tₘₐₓ of from about 1.5 hours to about 3.5 hours; (b) a mean plasma monomethyl fumarate Cₘₐₓ ranging from about 1.03 mg/L to about 3.4 mg/L; (c) a mean plasma monomethyl fumarate AUCₒᵥₑᵣₐₗₗ ranging from about 4.81 h·mg/L to about 11.2 h·mg/L; (d) a mean plasma monomethyl fumarate AUC₀₋₁₂ ranging from about 2.4 h·mg/L to about 5.5 h·mg/L; and (e) a mean AUC_{0-infinity} ranging from about 2.4 h·mg/L to about 5.6 h·mg/L.

30. The method of claim 29, wherein the composition is orally administered to the subject in need thereof.

31. The method of claim 30, wherein the compound that metabolizes to monomethyl fumarate is a compound of Formula I: 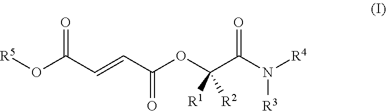
or a pharmaceutically acceptable salt thereof, wherein
R¹ and R² are independently chosen from hydrogen, C₁₋₆ alkyl, and substituted C₁₋₆ alkyl;
R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl; and
R⁵ is chosen from methyl, ethyl, and C₃₋₆alkyl;
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl;
with the proviso that when R⁵ is ethyl; then R³ and R⁴ are independently chosen from hydrogen, C₁₋₆ alkyl, and substituted C₁₋₆ alkyl.

32. The method of claim 30, wherein the compound that metabolizes to monomethyl fumarate is a compound of Formula II: 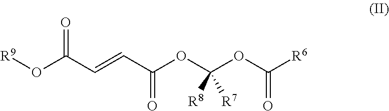
or a pharmaceutically acceptable salt thereof, wherein
R⁶ is chosen from C₁₋₆ alkyl, substituted C₁₋₆ alkyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl;
R⁷ and R⁸ are independently chosen from hydrogen, C₁₋₆ alkyl, and substituted C₁₋₆ alkyl; and
R⁹ is chosen from C₁₋₆ alkyl and substituted C₁₋₆ alkyl;
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.
